(19) United States
(12) Patent Application Publication
(10) Pub. No.: US 2012/0130198 A1
(43) Pub. Date: May 24, 2012

(54) SYSTEMS AND METHOD FOR DETERMINING AND MANAGING AN INDIVIDUAL AND PORTABLE HEALTH SCORE

(76) Inventor: Jean-Francois BEAULÉ, Farmington, CT (US)

(21) Appl. No.: 13/270,971

(22) Filed: Oct. 11, 2011

Related U.S. Application Data
(60) Provisional application No. 61/416,707, filed on Nov. 23, 2010.

(51) Int. Cl. A61B 5/00

(52) U.S. Cl. 600/300

(57) ABSTRACT

Methods and systems for determining and managing an individual and portable health score are disclosed. The method may include receiving individual health data. The method may further include determining one or more relevant health factors in response to the individual health data. Furthermore, the method may include assigning relative weights to the one or more relevant health factors. Subsequently, the method may include determining a baseline health score based on the relative weights of the one or more relevant health factors and the individual health data. The method for determining a health score may include adjusting the baseline health score in response to one or more qualified health actions, condition overrides, and/or quality checking. The disclosure may enable a national standard for a unique health score that reflects the combination of an individual's health status and the value of the individual's actions.

### CROSS-REFERENCE TO RELATED APPLICATIONS

This application claims priority to, and incorporates by reference in its entirety, U.S. Provisional Patent Application Ser. No. 61/416,707 entitled “Systems and Methods for Determining and Managing and Individual and Portable Health Score”, which was filed on Nov. 23, 2010.

### FIELD OF THE INVENTION

This invention relates to improving individual health and wellness and more particularly relates to systems and methods for determining and managing an individual and portable health score. Furthermore, this invention relates to systems and methods that connect and adapt to the health care industry.

### SUMMARY OF THE INVENTION

Embodiments of the methods and systems for determining and managing a individual and portable health score—also referred to in this disclosure as an individual's health score or simply “health score”—presented here disclose a unique health score that reflects the combination of an individual's health status and the value of the individual's actions. One overarching goal of an individual and portable health score may be to empower individuals with this information to provide motivation to improve their health. Individuals may be able to understand their health status relative to clinically accepted standards and be motivated to improve their health by taking certain actions (e.g., weight loss program). An individual may even be further motivated to improve his/her health by additional rewards incentives provided by the health insurance market.

In some embodiments, the methods and systems for determining health score measure standard biometrics that individuals have the ability to modify. It may be designed to inform and motivate individuals to be healthy and to proactively engaged with their health plan and the system as a whole. Embodiments of the health score may provide individuals: (1) a standard knowledge of the modifiable aspects of their health, that are common across all health plans; and (2) the motivation to engage in the health system to improve their health through compliance with evidence-based standards via a transparent scoring mechanism.

The health score may measure across a standardized scale recognizable by both the individual and the health care industry. In various embodiments, incremental improvements in health measures such as BMI, cholesterol and blood sugar may translate into positive increments on the scale. The design of a measure's weights and increments may be informed by the clinical and economic value of the measure itself. In preferred embodiments, the incremental approach is designed to deliver motivational value to the person.

Embodiments of the health score may also be integrated with a health benefits plan offering associated incentives to complete the compelling equation of individual health motivation. For example, in certain embodiments, anyone can maximize their score regardless of their health status. In some embodiment, consumers may benefit from their ability to “carry” the health status portion of their score with them wherever they go. This may allow them to be eligible for health benefit rewards as they change coverage, whether triggered by an employment or life-stage transition (e.g., entry into Medicare-based programs).

The methods and systems for determining and managing a personal and portable health score recognize the nature of a mobile work force. As such, the health score itself may be owned by the individual and move with the individual through different employers and/or health plans. The portability of a personal health score may drive the standardization of health quality measurements used by health plans, health care providers, government programs, and other players in the healthcare industry.

Methods and systems are disclosed. Methods for determining a health score are disclosed. In some embodiments, the methods may include receiving individual health data. In some embodiments, the methods may include determining one or more relevant health factors in response to the individual health data. In some embodiments, the methods may include assigning relative weights to the one or more relevant health factors. In some embodiments, the methods may include determining a baseline health score based on the relative weights of the one or more relevant health factors and the individual health data. In some embodiments, the methods may include adjusting the baseline health score in response to one or more qualified health actions.

In some embodiments, the methods may further include adjusting the baseline health score in response to one or more condition overrides. In some embodiments, the methods may further include adjusting the baseline health score in response to quality checking.

In some embodiments, determining one or more relevant health factors may include determining one or more core health factors. In some embodiments, determining one or more relevant health factors may include determining one or more age-gender based factors. In some embodiments, determining one or more relevant health factors may include determining one or more health condition based factors.

In some embodiments, determining the baseline health score may include determining a points adjustment for each relevant health factor in response to a result measurement and a target measurement. In some embodiments, determining the baseline health score may include subtracting the points adjustment for each relevant health factor from an initial health score.

In some embodiments, determining a points adjustment for each relevant health factor may include determining a miss metric by comparing the result measurement to the target measurement. In some embodiments, determining a points adjustment for each relevant health factor may include determining a final difference by comparing the miss metric to a metric cap. In some embodiments, determining a points adjustment for each relevant health factor may include determining a points per increment of the final difference by comparing the relevant health factor's relative weight to the metric cap. In some embodiments, determining a points adjustment for each relevant health factor may include determining the points adjustment by multiplying the final difference and the points per increment.

In some embodiments, adjusting the baseline health score in response to one or more certified qualified actions may include determining the applicability of a certified qualified action. In some embodiments, adjusting the baseline health score in response to one or more certified qualified actions may include determining the period of applicability of the certified qualified action. In some embodiments, adjusting the baseline health score in response to one or more certified qualified actions may include adjusting the baseline health score during the applicable period of the certified qualified action.

In some embodiments, adjusting the baseline health score in response to one or more condition overrides may include determining the applicability of a condition override. In some embodiments, adjusting the baseline health score in response to one or more condition overrides may include determining the period of applicability of the condition override. In some embodiments, adjusting the baseline health score in response to one or more condition overrides may include adjusting the incremental impact of one or more relevant health factors during the applicable period in response to the condition override.

In some embodiments, adjusting the baseline health score in response to quality checking may include receiving an appeal. In some embodiments, adjusting the baseline health score in response to quality checking may include determining the applicability of the appeal. In some embodiments, adjusting the baseline health score in response to quality checking may include determining the period of applicability of the appeal. In some embodiments, adjusting the baseline health score in response to quality checking may include adjusting the baseline health score during the applicable period of the appeal.

Systems for determining a health score are also disclosed. In some embodiments, the systems may include a data storage device configured to store a database comprising one or more records. In some embodiments, the systems may include a server in data communication with the data storage device suitably programmed. In some embodiments, the server may be suitably programmed to receive individual health data. In some embodiments, the server may be suitably programmed to determine one or more relevant health factors in response to the individual health data. In some embodiments, the server may be suitably programmed to assign relative weights to the one or more relevant health factors. In some embodiments, the server may be suitably programmed to determine a baseline health score based on the relative weights of the one or more relevant health factors and the individual health data. In some embodiments, the server may be suitably programmed to adjust the baseline health score in response to one or more qualified health actions.

In some embodiments, the server may further be suitably programmed to adjust the baseline health score in response to one or more condition overrides. In some embodiments, the server may be suitably programmed to adjust the baseline health score in response to quality checking.

Systems for managing health scores are also disclosed. In some embodiments, the systems may include a data storage device configured to store a database comprising one or more records. In some embodiments, the systems may include a server in data communication with the data storage device suitably programmed. In some embodiments, the server may be suitably programmed to receive one or more user inputs. In some embodiments, the server may be suitably programmed to receive healthcare data from one or more healthcare data sources in response to the one or more user inputs. In some embodiments, the server may be suitably programmed to aggregate the received healthcare data. In some embodiments, the server may be suitably programmed to determine the health score in response to the processed received user healthcare data.

In some embodiments, aggregating may include removing redundancy within the received healthcare data. In some embodiments, aggregating may include resolving anomalies within the received healthcare data.

In some embodiments, the one or more records may include received healthcare data. In some embodiments, the one or more records may include one or more health scores. In some embodiments, the one or more records may include one or more calculations used to determine the one or more health scores. In some embodiments, the one or more records may include one or more appeals.

In some embodiments, the one or more records may include a timestamp data describing when the records were stored in the data storage device. In some embodiments, the one or more records may include source data describing the source of the records.

In some embodiments, the server may be further programmed to output one or more records in response to one or more user inputs. In some embodiments, receiving one or more user inputs may include receiving one or more healthcare data source flow selections. In some embodiments, receiving one or more user inputs may include receiving one or more healthcare provider data flow selections. In some embodiments, receiving one or more user inputs may include receiving one more health plan data flow selections.

In some embodiments, the server may further be configured to control access to one or more records in response to receiving one or more health plan data flow selections.

In some embodiments, the server further configured to output one or more records to a health plan in response to one or more health plan data flow selections.

In some embodiments, receiving user healthcare data from one or more health data sources may include receiving user healthcare data across one or more data channels in response to one or more healthcare data source flow selections.

Additional methods are also disclosed. In some embodiments of the method, the method may include assigning relative weights to one or more core health factors, one or more age-gender based factors, and one or more health condition based factors. In some embodiments, the method may include determining a first intermediate health score in response to the one or more core factors. In some embodiments, the method may include determining a second intermediate health score by adjusting the first intermediate health score in response to the one or more age-gender based factors. In some embodiments, the method may include determining a third intermediate health score by adjusting the second intermediate health score in response to the one or more health condition based factors. In some embodiments, the method may include determining a fourth intermediate health score by adjusting the third intermediate health score in response to one or more condition overrides. In some embodiments, the method may include determining a fifth intermediate health score by adjusting the fourth intermediate health score in response to the one or more qualified health actions. In some embodiments, the method may include determining the health score by adjusting the fifth intermediate health score in response to quality checking.

The term “coupled” is defined as connected, although not necessarily directly, and not necessarily mechanically.

The terms “a” and “an” are defined as one or more unless this disclosure explicitly requires otherwise.

The term “substantially” and its variations are defined as being largely but not necessarily wholly what is specified as understood by one of ordinary skill in the art, and in one non-limiting embodiment “substantially” refers to ranges within 10%, preferably within 5%, more preferably within 1%, and most preferably within 0.5% of what is specified.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a method or device that “comprises,” “has,” “includes” or “contains” one or more steps or elements possesses those one or more steps or elements, but is not limited to possessing only those one or more elements. Likewise, a step of a method or an element of a device that “comprises,” “has,” “includes” or “contains” one or more features possesses those one or more features, but is not limited to possessing only those one or more features. Furthermore, a device or structure that is configured in a certain way is configured in at least that way, but may also be configured in ways that are not listed.

Other features and associated advantages will become apparent with reference to the following detailed description of specific embodiments in connection with the accompanying drawings.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 is a schematic flow chart diagram illustrating an embodiment of a method for determining an individual and portable health score in accordance with the present invention;

FIG. 2 is a schematic flow chart diagram illustrating an embodiment of a method for determining an individual and portable health score in accordance with the present invention;

FIG. 3 is a schematic flow chart diagram illustrating an embodiment of a method for adjusting an initial and/or intermediate health score in response to one or more health factors;

FIG. 4 is a schematic flow chart diagram illustrating an embodiment of a method for adjusting an intermediate health score to one or more condition overrides;

FIG. 5 is a schematic flow chart diagram illustrating an embodiment of a method for adjusting an intermediate health score in response to one or more qualified actions;

FIG. 6 is a schematic flow chart diagram illustrating an embodiment of a method for adjusting an intermediate health score in response to one or more quality checks;

FIG. 7 is a schematic block diagram illustrating one embodiment of a system for determining and/or managing an individual and portable health score;

FIG. 8 is a schematic block diagram illustrating one embodiment of a database system for determining and/or managing an individual and portable health score;

FIG. 9 is a schematic block diagram illustrating one embodiment of a computer system that may be used in accordance with certain embodiments of a system for determining and/or managing an individual and portable health score; and

FIG. 10 is a schematic block diagram illustrating one embodiment of a system that may be used in accordance with certain embodiments of a system for determining and managing an individual and portable health score.

### DETAILED DESCRIPTION

Various features and advantageous details are explained more fully with reference to the nonlimiting embodiments that are illustrated in the accompanying drawings and detailed in the following description. Descriptions of well known starting materials, processing techniques, components, and equipment are omitted so as not to unnecessarily obscure the invention in detail. It should be understood, however, that the detailed description and the specific examples, while indicating embodiments of the invention, are given by way of illustration only, and not by way of limitation. Various substitutions, modifications, additions, and/or rearrangements within the spirit and/or scope of the underlying inventive concept will become apparent to those skilled in the art from this disclosure.

In the following description, numerous specific details are provided, such as examples of programming, software modules, user selections, network transactions, database queries, database structures, hardware modules, hardware circuits, hardware chips, etc., to provide a thorough understanding of the present embodiments. One skilled in the relevant art will recognize, however, that the invention may be practiced without one or more of the specific details, or with other methods, components, materials, and so forth. In other instances, well-known structures, materials, or operations are not shown or described in detail to avoid obscuring aspects of the invention.

The schematic flow chart diagrams that follow are generally set forth as logical flow chart diagrams. As such, the depicted order and labeled steps are indicative of one embodiment of the presented method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

### Determining an Individual and Portable Health Score

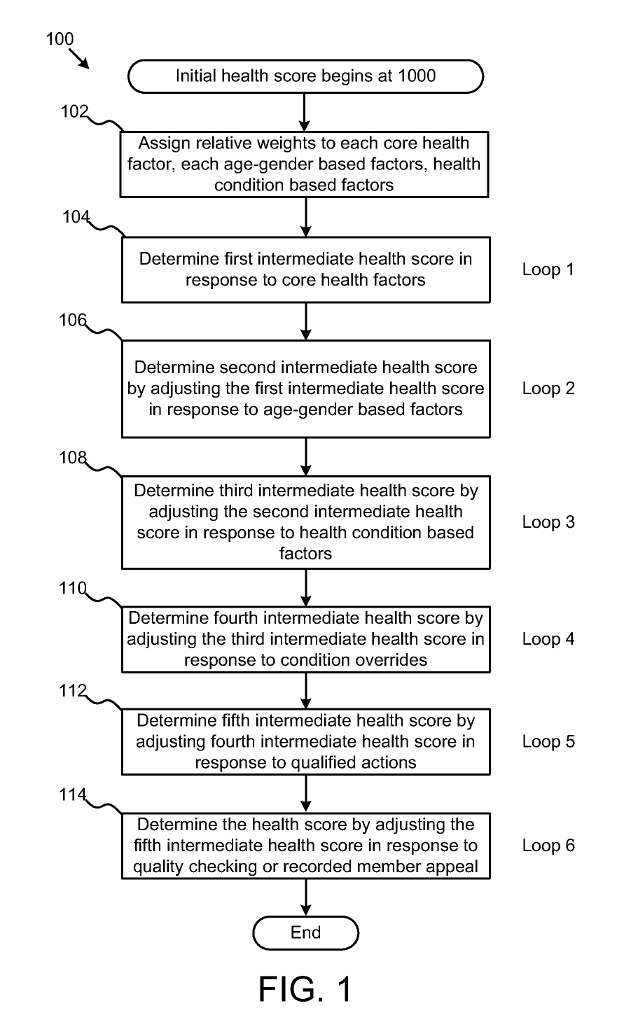

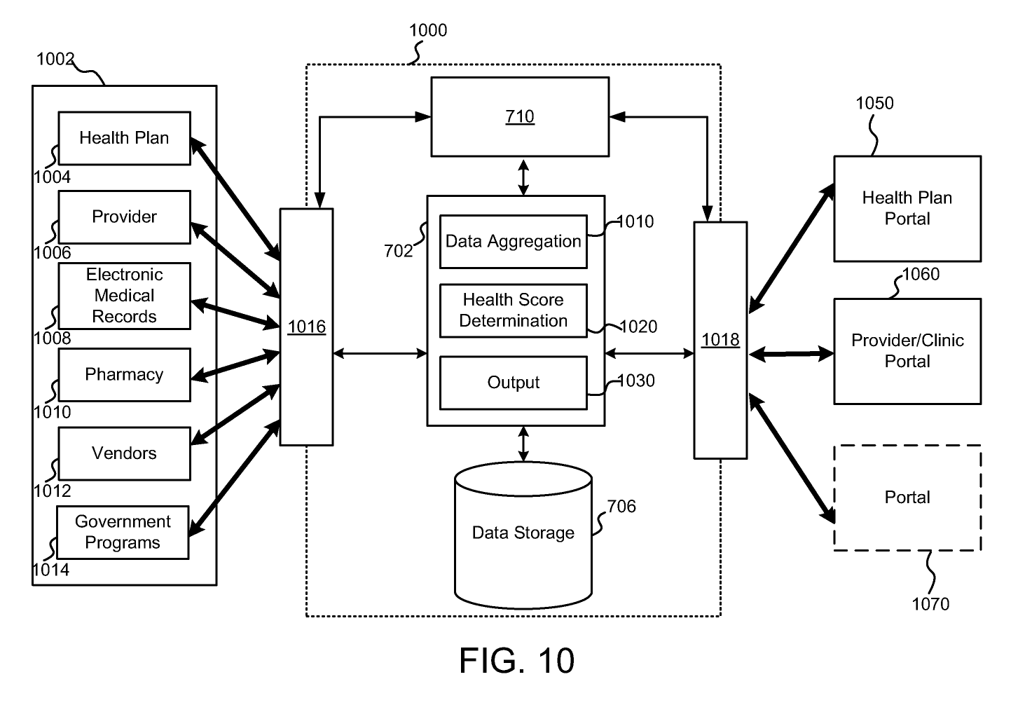

FIG. 1 illustrates one embodiment of a method 100 for determining an individual and portable health score. An individual's health score is determined based on that individual's received healthcare data. Individual health score data is not (in preferred embodiments) self-collected or self-reported. Rather, an individual's healthcare data includes objective biometric data compiled by trained and/or licensed medical practitioners and vendors. Such certification of biometric data lends greater credibility to the score once it is calculated. Even in an embodiments where healthcare data may be self-collected, such data may still be certified by trained and/or licensed medical practioners and vendors. An individual's healthcare data may include the individual's complete medical history and/or information gathered during a yearly doctor's visit. The collection and aggregation of healthcare data is described in more detail with regard to FIG. 10.

In some embodiments, an individual's initial health score begins at a fixed number or starting point (e.g., 1000). For example, in embodiments where an individual's health score begins at 1000, 1000 may represent the best attainable health score (e.g., health score ceiling) and 0 may represent the worst health score (e.g., health score floor). In some embodiments, the floor health score may be limited (e.g., 250). Subtracting the floor health score from the ceiling health score may reveal the total available health score points. For example, in an embodiment with a ceiling health score of 1000 and a floor health score of 250, an individual has 750 available health score points. Raising the floor health score (e.g., to 250 instead of 0) may help an individual utilize a health score without being discouraged because a lower floor health score may be a demotivator.

For example, in specific embodiments, a person with the maximum (ideal) health score of 1,000 could be one of three types of people:

(1) A person in good health who is taking proactive, preventive steps to stay healthy.

(2) A person whose core measures fall outside the ideal range, and who is at high risk of developing chronic illness in the future; however, they are actively enrolled in plan-sponsored programs that will help move their measures back to a healthier range. The health plan rewards that engagement by re-crediting the measures that are targeted by the program.

(3) A chronically ill person (with diabetes, CAD, COPD, hyperlipidemia or hypertension) who is fully compliant with their care regiment and either has stabilized their core health measures to the desired target or is engaged in health plan coaching programs to help improve those measures. As such, ideal health score may not always be associated with ideal health.

In some embodiments, the method 100 begins by assigning 102 relative weights to one or more health factors. These health factors include core health factors, age-gender based factors, and/or health condition based factors. These health factors may be grounded in well-accepted evidence-based medicine and quality guidelines—such as, for example, from the United States Preventive Services Task Force (USPTF) and/or the Healthcare Effectiveness Data and Information Set (HEDIS). The selection of the score components and their relative weights may be designed to provide a fully transparent and motivational framework that makes it easy for the individual to understand the aspects of their medium- and long-term health that they can change and improve upon. The explanation of various health factors—including core health factors, age-gender based factors, and condition based factors—that follows is provided without limitation. Moreover, the various health factors that may be considered and incorporated into a health score may evolve as new clinical evidences emerges.

### Core Health Factors

Core health factors are general, universal health metrics commonly used to indicate one's health. Core health factors may include measurements of weight, body mass index (BMI), waist line, body fat, smoker/non-smoker, blood glucose level, cholesterol levels (e.g., LDL, HDL, total cholesterol), blood pressure (e.g., systolic, diastolic), blood sugar (e.g., HbA1c/A1c), and other like measurements. In some embodiments, some or all of these core health factors are used to determine an individual's health score. For example, in a specific embodiment, BMI, smoking, blood glucose level, LDL, systolic blood pressure, and diastolic blood pressure measurements are used for the core health factors. Several core health measures that may be used in various embodiments of the health score are discussed in more detail below:

Tobacco Usage—According to the CDC, tobacco usage, which includes both the smoking and chewing of any tobacco-based product, is the leading preventable cause of death worldwide. Tobacco usage causes cancer, heart disease, stroke and lung disease, and is responsible for about one in five U.S. deaths every year. On average, smokers live 13-14 years less than nonsmokers. Cigarette smoking costs more than $193 billion (i.e., $97 billion in lost productivity plus $96 billion in health care expenditures). According to the CDC, tobacco usage, which includes both the smoking and chewing of any tobacco-based product, is the leading preventable cause of death worldwide. Tobacco usage causes cancer, heart disease, stroke and lung disease, and is responsible for about one in five U.S. deaths every year. On average, smokers live 13-14 years less than nonsmokers. Cigarette smoking costs more than $193 billion (i.e., $97 billion in lost productivity plus $96 billion in health care expenditures). Some embodiments of the health score consider the question of tobacco usage to be a binary response: non-tobacco usage would increase the health score, and tobacco usage would decrease the health score.

Body Mass Index (BMI)—BMI may be used as one metric to classify overweight and obese people. The Centers for Disease Control and Prevention (CDC) define overweight and obese people as follows:
An adult who has a BMI less than 18.5 is considered underweight.
An adult who has a BMI between 25 and 29.9 is considered overweight.
An adult who has a BMI of 30 or higher is considered obese.
An adult who has a BMI of 40 or higher is considered morbidly obese.

The USPSTF considers BMI to be reliable and valid for identifying adults at increased risk for mortality and morbidity due to being overweight and obese. The World Health Organization classifies obesity as a chronic disease. Obesity, particularly abdominal obesity, is correlated with the insulin resistance that is characteristic of type 2 diabetes. In fact, 9.2 percent of all obese people have diabetes, and 80 percent of diabetics are either obese or have a history of obesity.

According to the National Heart and Lung Institute, being overweight and obese puts people at high risk for developing other conditions such as: CAD, high blood pressure, gallstones, pulmonary issues, stroke, reproductive problems, cancer.

In some embodiments of the health score, a BMI of 25 or higher (or lower than 18.5) may be considered unhealthy. As discussed in more detail later in this disclosure, the health score may be reduced for decimal increments in an individual's BMI in an unhealthy range—(25.1, 25.2, etc.) up to a maximum BMI of 40.0 (morbidly obese). This approach may allow individuals to visually see improvements or deterioration in their score due to their weight while not creating an unnecessary sense of urgency when results are close to the target measurement. Such an approach may help mitigate issues like high/low bone or muscle density that can make the BMI measurement inaccurate for its targeted purpose.

Cholesterol—According to the CDC, having high cholesterol puts people at risk for cardiovascular disease, which can lead to heart attacks and strokes, the leading causes of death in the United States. High LDL cholesterol substantially increases the risk of heart disease. About one of every six adult Americans has high blood cholesterol. According to the American Heart Association, even though high cholesterol may lead to serious heart disease, most of the time there are no symptoms. This is why it is important for people to check their cholesterol levels on a regular basis.

The American Heart Association endorses the National Cholesterol Education Program (NCEP) guidelines for detection of high cholesterol, which includes a fasting lipoprotein profile or cholesterol screening. This screening measures the level of HDL and LDL in the bloodstream. HDL is the “good” cholesterol that helps keep the LDL, or “bad” cholesterol from getting lodged into the walls of the artery. A healthy level of HDL may also protect against heart attack and stroke, while low levels of HDL (less than 40 mg/dL for men and less than 50 mg/dL for women) have been shown to increase the risk of heart disease. The cholesterol screening report shows cholesterol levels in milligrams per deciliter of blood (mg/dL).

The USPSTF states that the optimal interval for screening is uncertain. On the basis of other guidelines and expert opinion, reasonable options include every five years, shorter intervals for people who have lipid levels close to those warranting therapy, and longer intervals for those not at increased risk who have had repeatedly normal lipid levels. It is also ambiguous about routine screening for adults over the age of 20 with no risk factors. USPSTF's recommendations for screening for high LDL cholesterol vary by age, risk factor and associated intervals.

Some embodiments of the health score only considers the LDL component of cholesterol in its calculation as it is very modifiable with the right medications, diet and exercise, and overall it is easier for people to understand. HDL can be very hard to change, even with medications. For example, the target measurement for LDL—above which the health score may be reduced—is 130 up to a maximum LDL level of 160 (integer increments). For individuals with a chronic illness like diabetes and CAD or health risks like hypertension, the target measurement may be reduced to 100.

In some embodiments, thus, the HDL metric has been left out of the score calculation. The metric may be recorded as a result to be shared with the individual. This will at least continue to support the dialogue fostered between the individual and their doctor regarding their overall cholesterol levels, including triglycerides. As clinical advancements and evidence emerges on both outcomes and the individual's ability to modify the results, other cholesterol measures would eventually be promoted into the scoring algorithms of the health score.

Blood pressure—High blood pressure, or hypertension, also increases the risk of heart attacks and stroke, which are the first- and third-leading causes of death among Americans. High blood pressure also can result in other conditions, such as congestive heart failure, kidney disease and blindness. Like high cholesterol, hypertension is a silent killer—it often has no warning signs or symptoms, and as such, should be monitored regularly. An estimated one in every three Americans currently has high blood pressure. About ⅔ of people over the age of 65 have high blood pressure. Hypertension is a condition that most people will have at some point in their lives.

Evidence is lacking recommending an optimal interval for screening adults for hypertension. The seventh report of the Joint National Committee on Prevention, Detection, Evaluation and Treatment of High Blood Pressure recommends screening:
Every 2 years in persons with blood pressure less than 120/80 mm Hg
Every year with systolic blood pressure of 120 to 139 mm Hg or diastolic blood pressure of 80 to 89 mm Hg.

In some embodiments of the health score, the target measurement of systolic blood pressure is 120. Higher than the 120 threshold, the health score may be incrementally reduced for blood pressures up to a maximum level of 160. In some embodiments, the diastolic level is not used in the calculation of the health score.

Blood sugar screening—A person's blood sugar level, also known as fasting blood glucose, is an indication of whether or not they have pre-diabetes or diabetes. According to a recent study by the Lewin Group, by 2020, an estimated 52 percent of Americans will have either pre-diabetes or diabetes. This has major implications for people's health and life expectancy. In addition, the study shows that an estimated $194 billion will be spent on diabetes-related care in 2010, and will rise to $500 billion by the year 2020.

Full-blown diabetes is also associated with high blood pressure and cholesterol and evidence shows that many adults can prevent the onset with controllable actions like weight loss. People with diabetes are at high-risk of developing micro-vascular complications like blindness, kidney damage and nerve damage as well as cardiovascular complications like heart attack and stroke.

Various embodiments of the health score accept three tests to measure blood sugar level: fasting blood glucose (target measurement<100), A1c test (target measurement<5.7), and two-house glucose (target measurement<140). If more than one result provided, the hierarchy will be: fasting blood glucose, A1c and, then, two-hour glucose. In some embodiments, for diagnosed diabetics, the health score only considers the A1c as a valid blood sugar test with a target measurement of less than 7 percent.

### Age-Gender Based Factors

Age-gender based factors may include those health metrics and diagnostics that are specific to an individual's age and/or gender. For example, age-gender based factors may include determining whether an individual has completed a physical, breast cancer screening (e.g., mammography), colorectal cancer screening (e.g., colonoscopy), cervical cancer screening, or prostate-specific antigen (PSA) test. An age-gender based factor may be age specific, gender specific, or both. For example, whether an individual has completed a physical is likely only an age specific health factor, but whether an individual has completed a mammography is likely both age and gender specific. As clinical evidence advances and industry practices warrant it, measures may be added or removed to reflect this evidence. As discussed with respect to the core health factors, in determining an individual's health score some or all of these age-gender based factors may be used.

In some embodiments, age-gender based screenings/services may be chosen for meeting the following criteria:
These services can prevent modifiable health diseases with a material incidence.
Individual compliance with these guidelines can substantially reduce the incidence of these cancers.
These services have demonstrated an impact on medical costs in the literature when viewed with the proper time horizon.
They all enable practical, effective data collection.

In addition to general physical examinations with biometric screening, a more details analysis of a few of these age-gender based factors is discussed below:

Cervical cancer screening—In 2007, about 12,000 women in the U.S. were diagnosed with cervical cancer, and about ⅓ of them died from it. While the incidence of cervical cancer is very low, it is a highly preventable, identifiable, and treatable condition with the availability of screening tests and HPV vaccines. When cervical cancer is found early, it is highly treatable and has good survival rates.

According to the Centers for Disease Control and Prevention (CDC), “all women are at risk for cervical cancer, but it is rare in women younger than 30 years of age.” The American College of Obstetricians and Gynecologists in their December 2009 “Clinical Management Guidelines for Cervical Cytology Screening” recommends that cervical cancer screening using cervical cytology tests (i.e., Pap smears) begin at age 21. It is recommended every two years for women aged 21-29, with either conventional or liquid-based cytology. Women aged 30 years and over who have had three consecutive Pap smear results that are negative for intraepithelial legions and malignancy may be screened every three years.

Women previously treated for CIN 2, CIN 3 or cancer, are at high risk of contracting cervical cancer in the future. Post-cancer medical regimen includes more frequent Pap smears than those recommended for women who have not contracted it.

In certain embodiments of the health score, females between the ages of 21 and 29 may be required to receive a Pap smear every two years. It may require women 30 and older to receive one every three years. In certain embodiments, the health score may have points deducted for women who have not received Pap smears according to this schedule.

Mammography—Breast cancer is the fifth leading cause of death for women in the U.S. In 2006, approximately 191,000 women were diagnosed with breast cancer, and about 41,000 women died from it. According to the CDC, “regular mammograms are the best tests doctors have to find breast cancer early, sometimes up to three years before it can be felt.” Early stage breast cancer is very treatable and many women go on to live long and healthy lives. Biennial mammograms can lower the risk of dying from breast cancer.

In certain embodiments, the health score may have points deduced for women between the ages of 50-74 who do not complete a biennial screening mammography.

Colorectal Cancer Screening—In 2007 (the most recent year numbers are available)—(1) 142,672 people in the United States were diagnosed with colorectal cancer, including 72,755 men and 69,917 women, and (2) 53,219 people in the United States died from colorectal cancer, including 27,004 men and 26,215 women.

According to the CDC, if men and women aged 50 or older had regular screening tests, as many as 60 percent of deaths from colorectal cancer could be prevented. Screening does two things: (1) it can find precancerous polyps so that they can be removed before turning into cancer, and (2) it also helps find colorectal cancer at an early stage, when it is highly treatable. The USPSTF recommended frequency of colorectal cancer screening varies according to the method used. Screening programs incorporating fecal occult blood testing, sigmoidoscopy or colonoscopy will all be effective in reducing mortality. Modeling evidence suggests that population screening programs between the ages of 50 and 75 years using either of the following regiments will be approximately equally effective in life-years gained, assuming 100 percent adherence to the same regimen for that period:

1. Sigmoidoscopy every five years combined with high-sensitivity fecal occult blood testing every three years

2. Screening colonoscopy at intervals of 10 years.

In embodiments of the health score, 50-75 year old individuals may be required to undergo either option 1 or 2 above; non-compliant individuals may be deducted points in their score.

### Health Condition Based Factors

Health condition based factors may be related to one or more health conditions and/or chronic illnesses. For example, an individual may be diagnosed with diabetes, coronary artery disease (CAD), hypertension, hyperlipidemia, asthma, congestive heart failure, (CHF), COPD, and/or other health conditions. Any given health condition may have one or more associated health condition based factors. For example, for an individual diagnosed with diabetes both the completion and the result of an A1C test, an eye exam, and/or creatinine test may each be condition based health factors. Similarly, for an individual diagnosed with CAD, the completion and the result of an ACE inhibitors test, beta blockers test, and other like metrics may each be condition based health factors. For an individual diagnosed with hypertension, whether that individual is taking a hypertension prescription may be a health condition based factor, and for an individual diagnosed with hyperlipidemia whether that individual is taking a specific type of prescription: statins, may be a health condition based factor. One of skill in the art will recognize several other conditions and several other condition-associated metrics that may be considered to be health condition based factors.

In some embodiments, individuals may be diagnosed with one of these health conditions and/or chronic illnesses in accordance with individual claims patterns (where claims coding (e.g., ICD-9) and pharmacy coding indicate an existing condition), biometric results evidenced in the core health measures (e.g., a given blood pressure may indicate hypertension), formal provider notification data (e.g., through appeals or other qualified processes for non-claims data input).

In some embodiments, the diagnosis of diseases such as diabetes, CAD and COPD remains associated with individuals for the rest of their lives, regardless of changes in their biometrics, as recommended by clinical guidelines. As such, the embodiments of the health score may reflect the persistence of the diagnosis and automatically adds the documented compliance measures and actions to the individual's scoring requirements on an annual basis.

### Calculating the Health Score

Assigning 102 relative weights to one or more health factors may include determining the relative importance of each health factor to an individual's health. For example, in some embodiments, each health factor—whether it be a core health factor, an age-gender based health factor, or a condition based health factor—could have an equal weight. For example, if 750 health score points are available to an individual with 10 relevant health factors, with equal weighting each health factor could affect the health score by up to 75 points. Based on well established wellness and industry standards, however, different health factors could have different weights. For example, a health factor that determines whether or not an individual smokes would likely be weighted higher than a health factor that determines whether or not an individual has completed a colonoscopy.

In some embodiments, assigning 102 relative weights to each core health factor, each age-gender based factor, and each health condition based factor may require determining whether a given health factor is relevant. For example, in most embodiments, each of the core health factors would likely be relevant to all individuals. That is, for most individuals, metrics related to weight, cholesterol, blood pressure, smoking and the like would be relevant to determine an individual's health score. On the other hand, certain age-gender based factors may only be relevant to certain groups of people. For example, whether an individual has completed a mammography would only be relevant to women of a certain age, and where an individual has completed a PSA test would only be relevant to men of a certain age. Moreover, whether an individual has completed a colonoscopy may only be relevant to men of a certain age and women of a different age. Similarly, certain condition based health factors may only be relevant to those individuals diagnosed with a given condition. Whether or not an individual has completed an A1C test may only be relevant to an individual diagnosed with diabetes—and would not likely be relevant to other individuals. Thus, in certain embodiments, assigning 102 relative weights to each core health factor, each age-gender based factor, and each health condition based factors means assigning relative weights to each relevant health factor based on an individual's health history.

Table 1 below provides one example of assigning 102 relative weights to one or more health factors for Individual A—a 45 year old male with no preexisting health conditions. For this example, there are 750 available points.

TABLE 1
Example of Assigning Relative Weights to
Relevant Health Factors for Individual A

| | | | Relative |
|---|---|---|---|
| | | Relative | Weight |
| | | Weight | Points |
| Core Health Factors | Weight (BMI) | 4 | 150 |
| | Smoking | 4 | 150 |
| | Glucose | 5 | 187.5 |
| | LDL/HDL | 2 | 75 |
| | BP—Systolic | 4 | 150 |
| | & Diastolic | | |
| | Subtotal | | 712.5 |
| Age-Gender Based | Physical | 1 | 37.5 |
| Health Factor | | | |

As shown in Table 1, Individual A has no relevant condition based health factors, one relevant age-gender based factor, and six relevant core health factors. The result of a glucose exam is the highest rated health factor with a relative weight of 5 and could potentially affect his health score by up to 187.5 points out of 750. Whether Individual A has completed a physical is the lowest weighted health factor with a relative weight of 1 and could potentially affect his health score by up to 37.5 points.

Table 2 below provides a second example of assigning 102 the relative weights to one or more health factors for Individual B—a 50 year old female with diabetes. For this example, also, there are 750 available points.

TABLE 2
Example of Assigning Relative Weights to
Relevant Health Factors for Individual B

| | | | Relative |
|---|---|---|---|
| | | Relative | Weight |
| | | Weight | Points |
| Core Health Factors | Weight (BMI) | 4 | 88.2 |
| | Smoking | 4 | 88.2 |
| | Glucose | 5 | 110.3 |
| | LDL/HDL | 2 | 44.1 |
| | BP—Systolic | 4 | 88.2 |
| | &Diastolic | | |
| | Subtotal | | 419 |
| Age-Gender Based | Physical | 1 | 22.1 |
| Health Factor | Mammography | 1 | 22.1 |
| | Colonoscopy | 1 | 22.1 |
| | Cervical | 1 | 22.1 |
| | Subtotal | | 88.4 |
| Condition Based | A1C—Complete | 2 | 44.1 |
| Health Factors | A1C—Result | 5 | 110.3 |
| | Eye exam | 2 | 44.1 |
| | Creatinine Test | 2 | 44.1 |
| | Subtotal | | 242.6 |

As shown in Table 2, Individual B has 4 relevant condition based health factors each related to diabetes. Individual B—based on her age and gender—also has 4 relevant age-gender based condition factors, as well as 6 relevant core health factors. The result of a glucose exam and the result of an A1C exam each make up the highest rated health factors with a relative weight of 5 and each could potentially affect her health score by up to 110.3 points out of 750. Whether Individual B has completed a physical exam, a mammography, a colonoscopy, or cervical exam are the lowest weighted health factors with a relative weight of 1 and each could potentially affect her health score by up to 37.5 points.

Individual A and Individual B, as used in Tables 1 and 2, respectively, are used as an example throughout the disclosure. As shown in Tables 1 and 2, because Individual A has fewer relevant health factors than Individual B, each of Individual A's health factors are weighted differently than each of Individual B's health factors. For example, the Weight Core health factor is weighted higher for Individual A than for Individual B. As more or less factors are considered, relative weights may change and may need to be redistributed.

In some embodiments, assigning 102 the relative weights of one or more health factors proceeds differently. As shown with respect to Tables 1 and 2, various health factors are weighted differently based on the specific factors relevant to a particular individual. In some embodiments, determining 102 the relative weights of the one or more health factors is not based on the relevance of any particular factor. Alternatively, in some embodiments, the relative weights of each health factor—whether a core health factor, age-gender based factor, or health condition based factor—has an assigned weight that remains consistent for all individuals regardless of age, gender, pre-existing conditions or the like.

TABLE 3
Example of Assigning Relative Weights to Health Factors

| | | | Relative |
|---|---|---|---|
| | | Relative | Weight |
| | | Weight | Points |
| Core Health Factors | Weight (BMI) | 2 | 200 |
| | Smoking | 2 | 200 |
| | Glucose | 2 | 200 |
| | LDL/HDL | 2 | 200 |
| | BP—Systolic | 2 | 200 |
| | & Diastolic | | |
| | Subtotal | | 1000 |
| Age-Gender Based | Physical | 1 | 100 |
| Health Factor | Mammography | 1 | 100 |
| | Colonoscopy | 1 | 100 |
| | Cervical | 1 | 100 |
| | Subtotal | | 400 |
| Condition Based | A1C Measurement | 1 | 100 |
| Health Factors | Eye exam | 1 | 100 |
| | Creatinine Test | 1 | 100 |
| | Subtotal | | 300 |

As shown above, with respect to Table 3, the following assigned relative weights and relative weight points may be used with any individual. As shown, each of the core health factors are rated equally and could affect any individuals score. Such an embodiment may provide a more simple distribution of relative weights. Since the relative weighting of various factors does not change over time, an individual may have a stronger grasp of how each of the health factors affects the overall health score. Moreover, such an embodiments may lead to a more predictable explanation of how points will be added or removed, and as such, individuals more easily and reliably track changes to their health score.

In some embodiments, the method 100 further includes determining 104 the first intermediate health score in response to the core health factors. Determining 104 the first intermediate health score may begin by determining a points adjustment for each relevant core health factor and subtracting the points adjustment for each factor from the initial health score. For those factors that necessitate completion of a test or exam, failure to complete the test or exam may result in a points adjustment that corresponds to the full weight of the health factor, and for those factors that are numeric metrics the points adjustment may proportionately be based on the result of the measurement compared to an ideal. In some embodiments, the health score for an individual has a potential for being lower than the floor. For example, with respect table 3, a very unhealthy individual can have as many as 1000 points deducted simply based on her core health factors—that is the gross value of the points deducted from the health score for the five core health factors is 1000. In an embodiment where the health score begins at 1000 and has a floor at 250, such a health score may be “floored” to a value of 250. As discussed in more detail with respect to the clinical studies of Tables 8 and 9, only 2% of the population had a health score below the 250 floor in the studies.

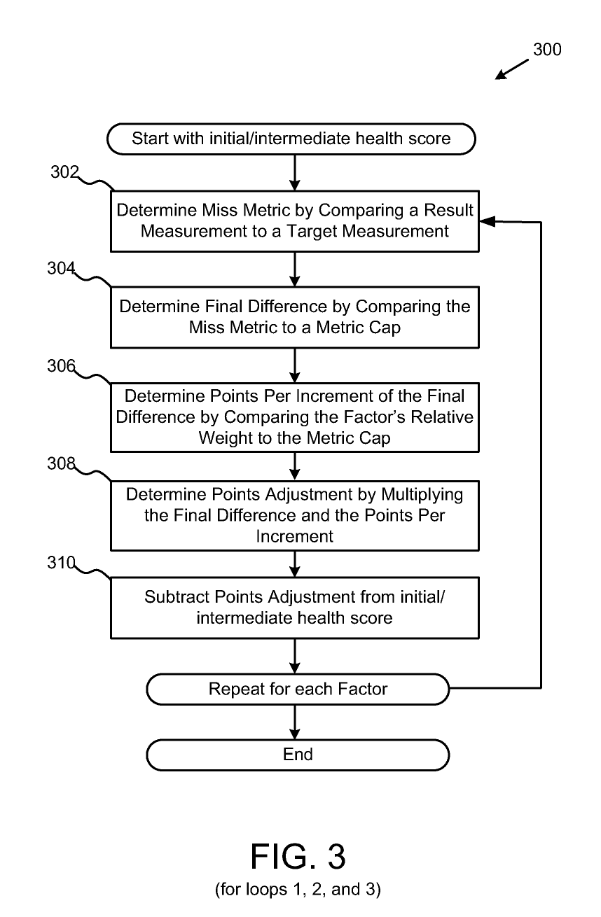

For example, as discussed with regard to Table 1, if Individual A is a smoker, his initial health score could be reduced by a point adjustment of up to 88.2 points. If Individual A is not a smoker, his initial health score would not be affected. For a numeric metric, like for example BMI, if Individual A has an ideal BMI, his health score would not be affected. If Individual A has an unhealthy BMI (e.g., too high or too low), that Individual A's initial health score may be reduced by a point adjustment proportionately based on how unhealthy his BMI actually is—the more unhealthy, the greater the points adjustment. Thus, if Individual A has an extremely unhealthy BMI, his initial health score may be reduced by a points adjustment of up to 88.2 points. In some embodiments, each health factor may be similarly analyzed and its impact (if any) may be subtracted from the initial health score to determine a first intermediate health score. Specific embodiments for determining an intermediate health score are discussed in more detail with regard to FIG. 3.

Similarly, the method 100 may further include determining 106 the second intermediate health score by adjusting the first intermediate health score in response to age-gender based factors. For example, similar to the discussion of core health factors, if an individual has completed his yearly physical, his or health score would not be affected. On other hand, if an individual has not complete his yearly physical the first intermediate health score may be reduced/decremented based on that health factor.

In some embodiments, the method 100 may further include determining 108 the third intermediate health score by adjusting the second intermediate health score in response to the health condition based factors. In some embodiments, the third intermediate health score is calculated in much the same way as described before: for those factors that necessitate completion of a test or exam, failure to complete the test or exam may result in a points adjustment that corresponds to the full weight of the health factor, and for those factors that are numeric metrics the points adjustment may proportionately be based on the result of the measurement compared to an ideal.

In some embodiments, the third intermediate health score may also be referred to as the “baseline health score.” The baseline health score may reflect the overall health of the individual based on all the relevant health factors, but does not take into account condition overrides, qualified actions, and quality checking/appeals.

In some embodiments, method steps 102, 104, 106, and 108 may be used to calculate the baseline health score may proceed sequentially as described or in a different order. In other embodiments, method steps 104, 106, and 108 where intermediate health scores are calculated may proceed simultaneously—in other words the core health factors, age-gender based factors, and condition based factors may be grouped together. In such embodiments, the baseline health score may be calculated directly from the initial health score without the calculation of intermediate health scores.

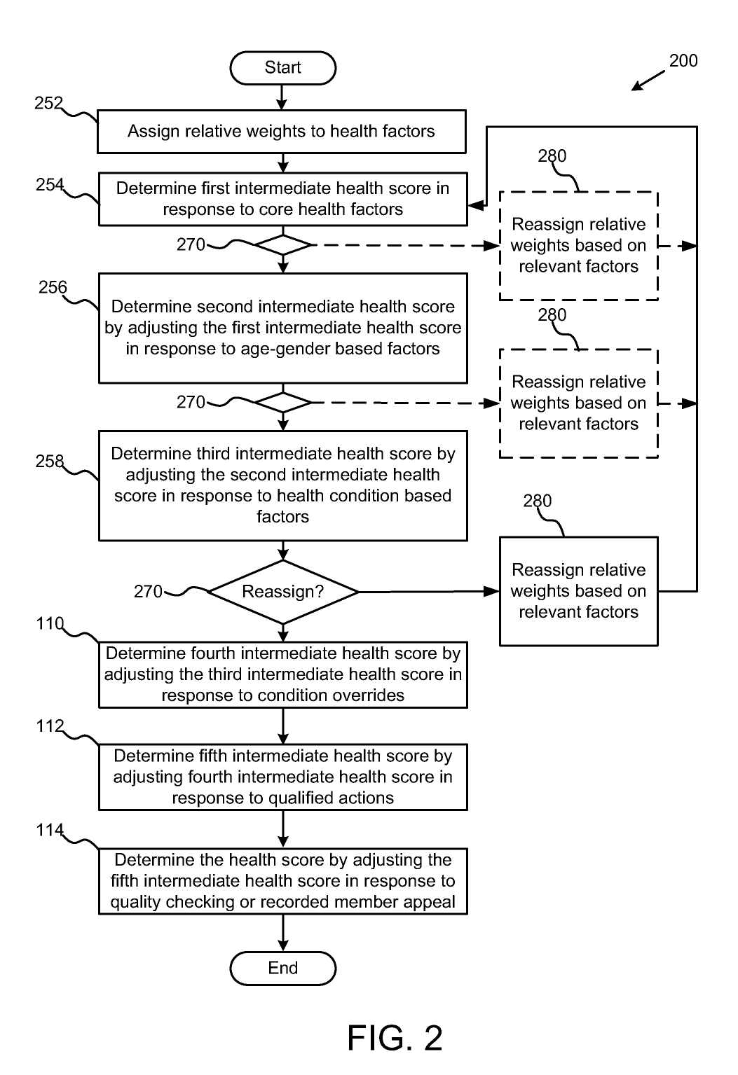

FIG. 2. illustrates an embodiment of a method 200 for determining the baseline health score. In some embodiments, relevant health factors for an individual may change over time. For example, as an individual ages, certain age-gender based factors may become relevant and/or irrelevant. An individual might be diagnosed with a disease, and thus certain condition based factors may become relevant and/or irrelevant. As health factors become relevant (or irrelevant) the relative weighting between health factors may change.

The method 200 may begin by assigning 252 relevant weights to the relevant health factors. The method 200 may proceed by determining 254 a first intermediate health score, determining 256 a second intermediate health score, and determining 258 a third intermediate health score in much the same way as described with regard to method steps 104, 106, and 108. After determining each intermediate health score, the method 200 may determine 270 whether relative weights of relevant health factors need to be assigned and/or reassigned. As such if new relevant health factors need to be considered (or old factors no longer need to be considered) in determining a health score, the relative weights of all health factors may need to be reassigned 280. Thus as shown in FIG. 2, if the relative weights of relevant health factors are reassigned 280, the intermediate health scores may need to be determined again—and thus steps 254, 256, and 258 may need to be repeated. As discussed with respect to Table 3, however, in some embodiments, the relative weighting between various health factors may not change overtime—regardless of the relevancy of certain health factors.

FIG. 3 illustrates an embodiment of method 300 for determining an intermediate health score by adjusting an initial/intermediate health score in response to one or more health factors. In some embodiments, the steps of method 300 may be used to complete method steps 104, 106, and 108 of method 100. As discussed earlier, determining an intermediate health score may require determining a points adjustment for each health factor and subtracting the points adjustment from an initial/intermediate health score.

The method 300 may begin with an initial or intermediate health score. In some embodiments, the method 300 continues by determining 302 a miss metric by comparing a result measurement to a target measurement. In some embodiments, the result measurement corresponds to an actual measurement of a particular health factor based on an individual's biometric data, and the target measurement corresponds to an ideal measurement of a particular health factor based on well-established industry standards. In some embodiments, the miss metric is the absolute value of the difference between the target measurement and the result measurement.

Table 4 provides an example of determining 302 a miss metric by comparing the result measurement to a target measurement for Individual A and Individual B for the BMI core health factor. As shown in Table 4, the target measurement (e.g., ideal) is 25. Individual A's result measurement (e.g., actual BMI measurement) is 33, and therefore his miss metric is 8. Individual B's result measurement is 29, and therefore her miss metric is 4. (See Tables 1 and 2 for more metrics regarding Individual A and Individual B, respectively).

TABLE 4
Determining a Points Adjustment in Response to BMI

| | Result | Target | Miss | Metric | Final | Points Per | Points |
|---|---|---|---|---|---|---|---|
| | Measurement | Measurement | Metric | Cap | Difference | Increment | Adjustment |
| Individual | 33 | 25 | 8 | 15 | 8 | 10 | 80 |
| A | | | | | | | |
| Individual | 29 | 25 | 4 | 15 | 4 | 5.88 | 23.52 |
| B | | | | | | | |

The method 300 may continue by determining 304 a final difference by comparing the miss metric to a metric cap. Determining 304 a final difference may help cap or limit the points adjustment associated with a given health factor. Metric caps are determined on an individual health factor basis based on the combination of well established industry health standards and the statistical likelihood of people having a higher/lower result. For BMI, for example, the metric cap may be 15. Therefore, if an individual has a BMI miss metric greater than 15—and thus a BMI greater than 40, that individual's final difference would be capped at 15. In some embodiments, the metric cap determines how much the final difference can vary (e.g., the range) for a given health factor. For this example, an individual's BMI final difference may vary from 0 to 15. In some embodiments, the metric cap may be limited so that the range of the final difference may capture a coverage of 95-97 percent of the population for that given metric.

For example, the miss metric for Individual A is 8 and that is less than the metric cap of 15. Similarly, the miss metric for Individual B is 4 and that is also less than the metric cap of 15. Therefore, the final difference for each individual corresponds to the miss metric. If, however, an individual had a BMI result measurement of 45, and therefore a miss metric of 20, that individual's final difference would be capped at 15.

The method 300 may also include determining 306 a points per increment of the final difference by comparing the relevant health factor's relative weight to the metric cap. As discussed earlier, a health factor's relative weight may determine how many health score points that health factor could affect. Referring back to Table 1 and Table 2, for example, for Individual A, it was determined that the BMI health factor could potentially affect his health score by up to 150 points, and for Individual B, it was determined that the BMI health factor could potentially affect her health score by up to 88.2 points. Furthermore, a health factor's metric cap may determine the range of the final difference. For example, for BMI, it was determined that the BMI final difference may vary from 0 to 15. In some embodiments, dividing the health factor's relative weight points by the metric cap provides the points per increment of the final difference. For example, for Individual A, dividing 150 by 15 provides a points per increment of the final difference of 10, and for Individual B, dividing 88.2 by 15 provides a points per increment of the final difference of 5.88.

As discussed earlier, the metric cap may determine the range of the final difference. A metric cap may be carefully calibrated so that it is neither too low or too high. Having a lower metric cap enables for a meaningful points per increment number—the smaller the range of the final different, the large the points per increment. As such, if individuals improve their health, the improvement may be reflected in their health score. If the metric cap is too low, however, individual improvements for a given metric may not be reflected in the health score because individual improvements may be outside the range of the final difference.

In some embodiments, the method 300 may also include determining 308 the points adjustment for a health factor by multiplying the final difference and the points per increment of the final difference. Referring to Table 4, for Individual A, multiplying the final difference and points per increment results in a points adjustment of 80 for the BMI health factor. For Individual B, the points adjustment for the BMI health factor is 23.52 (or rounded to 24).

In some embodiments, the method 310 may also include subtracting 310 the points adjustment for the health factor from the initial or intermediate health score. Thus, for Individual A, his initial health score will be reduced from 1000 to 920 based on the BMI health factor, and for Individual B her initial health score will be reduced from 1000 to 976. To complete the calculation of a baseline health score, the method steps 302, 304, 306, 308, and 310 may be repeated for each relevant health factor.

### Condition Overrides

Referring back to FIG. 1, after determining a baseline health score, in some embodiments, the method 100 may include determining 110 a fourth intermediate health score by adjusting the third intermediate health score in response to condition overrides. In some embodiments, the method may include adjusting the baseline health score in response to condition overrides. In some embodiments, a condition override may allow an individual to earn back health score points as a result of a particular short term condition or ailment. Condition overrides may exist for pregnancy, cancer, or other such severe or high risk conditions. In some embodiments, a physician may be able to manually apply a condition override based on specific metrics or observations that she has seen.

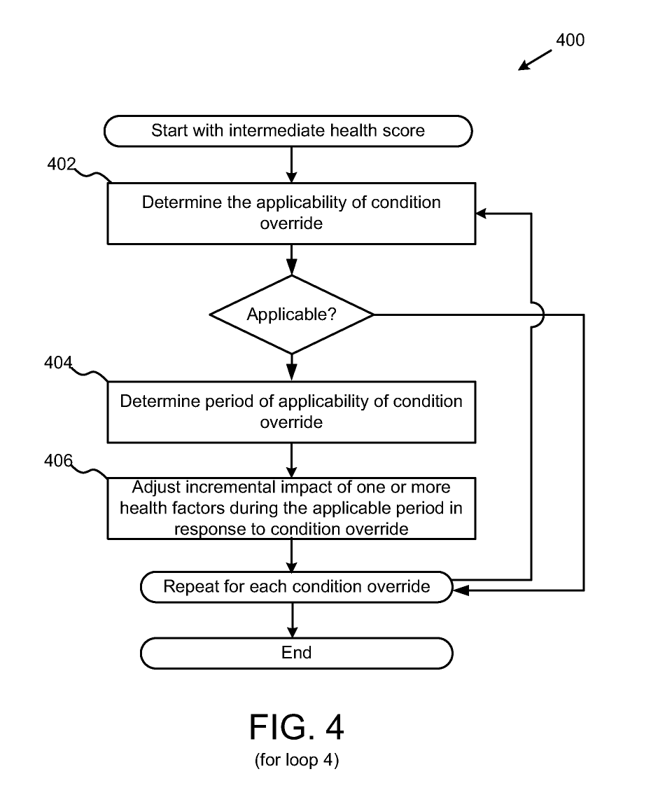

FIG. 4 provides an embodiment of a method 400 for adjusting an intermediate health score in response to a condition override. In some embodiments, the method may begin by determining 402 the applicability of a given condition override. As discussed earlier, only certain conditions—such as pregnancy and cancer—and physician overrides can suitably serve as a condition override. If a given condition is not applicable for an override, the method 400 moves on to the next condition override. If a given condition is applicable, the method 400 continues by determining 404 the applicable period of the condition override. For example, the applicable period for a pregnancy condition override may be the time during pregnancy and up to 3 months after pregnancy. Each applicable condition may have its own applicable period based on well established health and wellness standards.

In some embodiments, the method 400 may also include adjusting 406 the incremental impact of one or more health factors during the applicable period. Condition overrides may be tied to specific targeted metrics that relate to a specific condition. For example, the pregnancy condition override may specifically be tied to BMI—a pregnant woman is naturally expected to have a higher than ideal BMI during and after her pregnancy. As such if the BMI health factor adjusted an individual's health score that adjustment may be negated. Moreover, if as a result of the BMI health factor an individual's health score was reduced by 24 points, the pregnancy condition override may add back some or all of the 24 point reduction to the health score. In a different example, the pregnancy condition override may allow the preservation of the value of the last recorded pre-pregnancy BMI. As such, during a qualified period (e.g., up to the delivery of a child plus a postpartum period), the change caused by a condition (e.g., pregnancy or other qualified condition discussed above) may not affect an individual's health score. Providers and sponsors of qualified actions may be able to assist individuals understand how condition overrides may affect their health score, and in some instances may be provide incentives (e.g., rewards or health score adjustment) to motivate individuals to improve their health score. The method steps 402, 404, and 406 may be repeated for each condition override.

### Qualified Actions

Referring back to FIG. 1, after determining a baseline health score, in some embodiments, the method 100 may include determining 112 a fifth intermediate health score by adjusting the fourth intermediate health score in response to a qualified action. In some embodiments, the method may include adjusting the baseline health score in response to a qualified action. A qualified action is an action taken by an individual to improve his or her health. Such a qualified action would be reviewed against well established, certified health and wellness standards. Examples of qualified actions include attending a quit-smoking clinic, taking part in a weight-loss program, regular exercise at a certified gym (e.g., YMCA), or the like. In some embodiments, a qualified action may include maintenance of objective biometric data. Individuals may be required to update their biometric data every so often (e.g., once per year). For example, biometric data may be updated with a simple yearly checkup. In some embodiments, if biometric data is kept up to date an individual may receive a additional points, and if biometric data is not kept up to date, an individual may receive a points deduction.

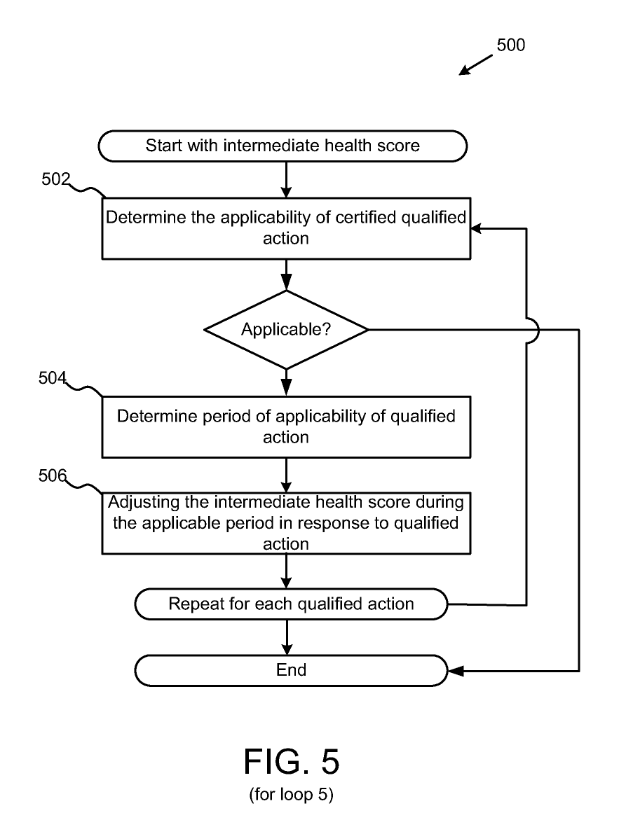

FIG. 5 provides an embodiment of a method 500 for adjusting an intermediate health score in response to a qualified action. In some embodiments, the method may begin by determining 502 the applicability of a given qualified action. As discussed earlier, only certain qualified actions are certified. In some embodiments, certification of a qualified action may have two parts. First, a process may be administered to ensure that a given qualified action provider actually provides a certified qualified action. In some embodiments, not every qualified action provider (e.g., gym, health coach, website) may be able to administer a qualified action. As such, only those providers whose programs have been properly reviewed and vetted may administer a qualified action. Second, the qualified action provider may further verify the engagement of the individual. The provider may determine whether the individual successfully completed the qualified action.

Providers and sponsors of qualified actions may be able to assist individuals complete the qualified actions and understand how completion of a qualified action may help boost an individual's health score. The intention behind the qualified action is to help motivate individuals to improve their score by completing these qualified programs. Though an individual may get a certain amount of action points added to their score for completing a smoking cessation program, the action points are not intended to reflect that the individual has “cured” the negative effects of smoking. Rather, the action points reflect that the individual is actively trying to improve their health. Significantly, adjustments to the health score are not performed arbitrarily. Rather, action points may be added to a health score during a qualified period based on a specific program, qualified action, condition override, appeal, and/or other similar means.

In some embodiments of the method 500, a qualified action provider may lose its certification. For example, if a provider fails to maintain proper standards, its certification may be eliminated. Additionally, a given provider's program may lose its certification if the individuals within their program do not actually improve their health (e.g. by demonstrating an improvement in one or more health factors as a result of the qualified action).

If a given qualified action is not certified, the method 500 moves on to the next qualified action. If a given qualified action is certified, the method 500 continues by determining 504 the applicable period of the qualified action. For example, the applicable period for a qualified action for completing a non-smoking clinic may be applicable for a fixed time period (e.g., 6 months) after completing the clinic. Each qualified action may have its own applicable period based on well established health and wellness standards.

In some embodiments, the method 500 may also include adjusting 506 the intermediate/baseline health score during the applicable period of the certified qualified action. As discussed earlier, these adjustments may include incremental action points or disincentive added to or subtracted from an intermediate health score. The method steps 502, 504, and 506 may be repeated for each qualified action.

### Quality Checking

Referring back to FIG. 1, after determining a baseline health score, in some embodiments, the method 100 may include determining 114 the final health score by adjusting the fifth intermediate health score in response to a quality checking. In some embodiments, the method may include adjusting the baseline health score in response to quality checking. Quality checking may include manual and/or automated systems to check for discrepancies and or variations in an individual's health score. An example of quality checking may be a user-submitted appeal. For example, an individual may notice an error in his or her health score and submit an appeal to get the error corrected—a piece of biometric data could be incorrect or the completion of a qualified action could be overlooked. In some embodiments, an agent of the user may submit an appeal on behalf of the individual.

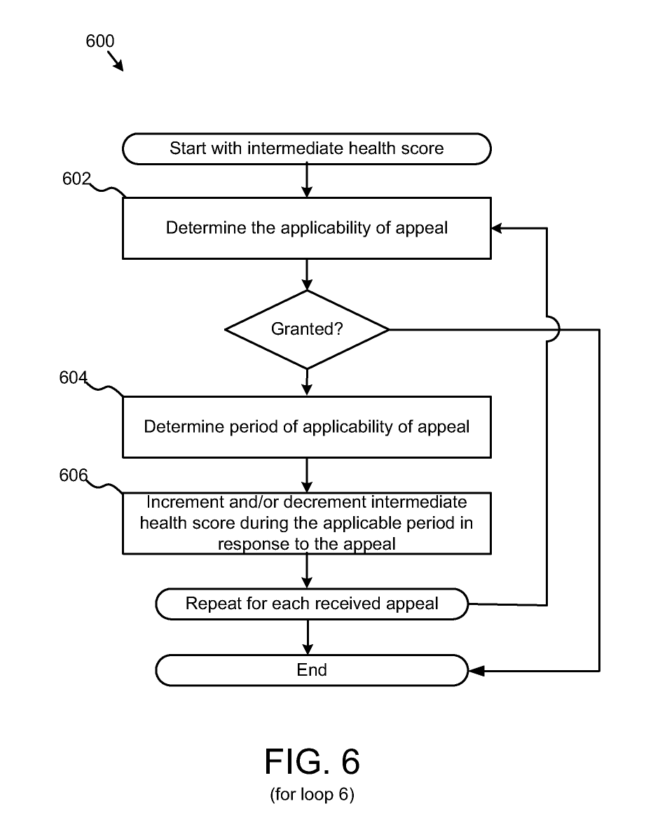

FIG. 6 provides an embodiment of a method 600 for adjusting an intermediate health score in response to an appeal. In some embodiments, the method may begin by determining 602 the applicability of a given appeal. As is expected, certain appeals may be granted and others may be dismissed. If a given qualified action is not granted, the method 600 moves on to the next appeal. If a given appeal is granted, the method 600 continues by determining 604 the applicable period of the appeal. In some embodiments, the method 600 may also include adjusting 606 the intermediate/baseline health score during the applicable period of the certified qualified action. Depending on the type of appeal and the change requested, the baseline/intermediate health score may be adjusted 606 accordingly.

Protocols and systems regarding condition overrides, qualified actions, and appeals are discussed in more detail with respect to FIG. 10.

Table 5 below provides an example of one embodiment for determining a health score for Individual A (refer back to Tables 1 and 3 for more information regarding Individual A):

TABLE 5
Individual A, Determining Health Score

| | | |
|---|---|---|
| | Initial Health Score | 1000 |
| Core Health Factors | Weight (BMI) | −80 |
| | Smoking | −150 |
| | Glucose | −24 |
| | LDL/HDL | −38 |
| | BP—Systolic | −18 |
| | & Diastolic | |
| | First Intermediate Health Score | 690 |
| Age-Gender Based | Physical | 0 |
| Health Factor | | |
| | Second Intermediate Health Score | 690 |
| Health Condition | N/A | 0 |
| Based Health Factor | | |
| | Third Intermediate (Baseline) | 690 |
| | Health Score | |
| Condition Overrides | N/A | 0 |
| | Fourth Intermediate Health Score | 690 |
| Qualified Actions | Smoking Cessation Program | 150 |
| | Fifth Intermediate Health Score | 840 |
| Quality Checking/Appeals | N/A | 0 |
| | Final Health Score | 840 |

As described in Table 5, Individual A's initial health score begins at 1000. As a result of the relevant core health factors, Individual A's initial health score is adjusted to 690. An analysis of each core health factor reduced his health score by various points adjustments. Individual A completed his required physical, and as a result of this age-gender based health factor, Individual A's first intermediate health score was not adjusted. For Individual A, health condition based health factors and condition overrides were not applicable—none were relevant to him. As shown his fifth intermediate health score increases the prior intermediate health score to 840. Individual A has completed a smoking cessation program qualified action. By completing this program, Individual A has added back points to his overall score. No appeals were relevant, and thus Individual A's final health score at this point in time is 840.

Table 6 below provides an additional example of determining a health score for Individual B (refer back to Tables 3 and 4 for more information regarding Individual B).

TABLE 6
Individual B, Determining Health Score

| | | |
|---|---|---|
| | Initial Health Score | 1000 |
| Core Health Factors | Weight (BMI) | −24 |
| | Smoking | 0 |
| | Glucose | −90 |
| | LDL/HDL | −32 |
| | BP—Systolic | −58 |
| | & Diastolic | |
| | First Intermediate Health Score | 796 |
| Age-Gender Based | Physical | 0 |
| Health Factor | Mammography | −22 |
| | Colonoscopy | 0 |
| | Cervical | −22 |
| | Second Intermediate Health Score | 752 |
| Health Condition | A1C—Complete | 0 |
| Based Health Factors | A1C—Result | −60 |
| | Eye exam | 0 |
| | Creatinine Test | −44 |
| | Third Intermediate (Baseline) | 648 |
| | Health Score | |
| Condition Overrides | N/A | 0 |
| | Fourth Intermediate Health Score | 648 |
| Qualified Actions | N/A | 0 |
| | Participation in Diabetic | 203 |
| | Disease Management Program | |
| | Fifth Intermediate Health Score | 851 |
| Quality Checking | N/A | 0 |
| | Final Health Score | 851 |

As shown in Table 6, condition overrides and quality checking did not affect Individual B's health score. Individual B did complete a certified Diabetic Disease Management Program. In this embodiment, completion of the qualified action allowed Individual B to add back 203 points to her health score. Specifically, for Individual B 90 points had previously been subtracted from her score based on her glucose score, 32 points based on her cholesterol, 21 points based on her blood pressure, and 60 points based on her A1C-Result. These 203 points are added back by successfully completing this certified qualified action. In some embodiments, the adding back of points may only be reflected in the health score for a certain pre-determined period of time after which the individual would have to re-test to see if their results actually improve. For example, if 6 months after completing the Diabetic Disease Management Program, Individual B's glucose and/or A1C levels did not improve, some or all of the 150 points originally added back to the score may be removed. Thus, Individual B's baseline health score of 851 remained Individual B's final health score.

Table 7 below provides an additional example of determining a health score for Individual A. The health score is Table 7 is calculated with respect to the weighting identified in Table 3.

TABLE 7
Individual A & B, Determining Health
Score with Different Weighting

| | | Individual | Individual |
|---|---|---|---|
| | | A | B |
| | Initial Health Score | 1000 | 1000 |
| Core Health Factors | Weight (BMI) | −107 | −54 |
| | Smoking | −200 | 0 |
| | Glucose (A1C for | −44 | −39 |
| | Diabetics) | | |
| | LDL/HDL | −86 | −79 |
| | BP—Systolic | −101 | −168 |
| | & Diastolic | | |
| | First Intermediate | 462 | 660 |
| | Health Score | | |
| Age-Gender Based | Physical | 0 | 0 |
| Health Factor | Mammography | 0 | −100 |
| | Colonoscopy | 0 | 0 |
| | Cervical | 0 | −100 |
| | Second Intermediate | 462 | 460 |
| | Health Score | | |
| Health Condition Based | A1C Measurement | 0 | 0 |
| Health Factors | Eye exam | 0 | 0 |
| | Creatinine Test | 0 | −100 |
| | Third Intermediate | 462 | 360 |
| | (Baseline) Health | | |
| | Score | | |
| Condition Overrides | N/A | 0 | 0 |
| | Fourth Intermediate | 462 | 360 |
| | Health Score | | |
| Qualified Actions | Diabetic Disease | 0 | 286 |
| | Management Program | | |
| | Smoking Cessation | 200 | 0 |
| | Program | | |
| | Fifth Intermediate | 662 | 646 |
| | Health Score | | |
| Quality Checking | N/A | 0 | 0 |
| | Final Health Score | 662 | 646 |

As shown in Table 7, as a result of the different weighting applied, Individuals A & B actually have different health scores when calculated with respect to the weighting of Table 3 instead of Tables 1 or 2. The embodiment shown with respect to Table 7 is a preferred embodiment that may enable an individual to see how their health score changes over time because, in this embodiment, the relative weights of the health factors remain constant.

Though they have been described sequentially, method steps 110, 112, and 114 may occur in any order. As implied above, in some embodiments, these method steps may not be relevant to each individual. Moreover, in some embodiments, each of the method steps of method 100 may be repeated individually as new healthcare data information is received for an individual.

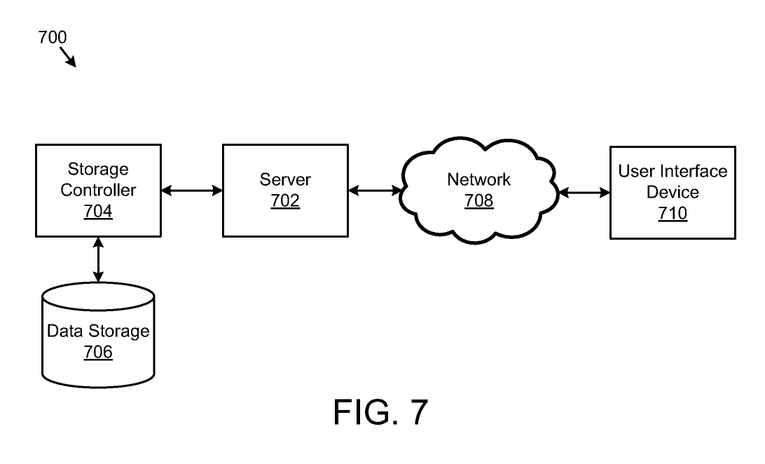

FIG. 7 illustrates one embodiment of a system 700 for determining a individual and portable health score. The system 700 may include a server 702, a data storage device 704, a network 708, and a user interface device 710. In a further embodiment, the system 700 may include a storage controller 706, or storage server configured to manage data communications between the data storage device 704, and the server 702 or other components in communication with the network 708. In an alternative embodiment, the storage controller 706 may be coupled to the network 708. Specifically, the system 700 may configured to store healthcare data for one or more individuals, determine a health score for one or more individuals, and store a health score (and intermediate health scores) for one or more individuals.

In one embodiment, the user interface device 710 is referred to broadly and is intended to encompass a suitable processor-based device such as a desktop computer, a laptop computer, a Personal Digital Assistant (PDA), a mobile communication device, tablet computer, or organizer device having access to the network 708. In a further embodiment, the user interface device 710 may access the Internet to access a web application or web service hosted by the server 702 and provide a user interface for enabling a user to enter or receive information. For example, in certain embodiments, an individual may be able to view his or her health score determination and examine the effects of each of the various health factors, qualified actions, condition overrides, and appeals.

The network 708 may facilitate communications of data between the server 702 and the user interface device 710. The network 708 may include any type of communications network including, but not limited to, a direct PC to PC connection, a local area network (LAN), a wide area network (WAN), a modem to modem connection, the Internet, a combination of the above, or any other communications network now known or later developed within the networking arts permits two or more computers to communicate, one with another.

In one embodiment, the server 702 is configured to receive individual health data, determine one or more relevant health factors in response to the individual health data, assign relative weights to the one or more relevant health factors, determine a baseline health score based on the relative weights of the one or more relevant health factors and the individual health data, and adjust the baseline health score in response to one or more qualified health actions, condition overrides, and/or quality checks. Additionally, the server may access data stored in the data storage device 104 via a Storage Area Network (SAN) connection, a LAN, a data bus, or the like.

The data storage device 704 may include a hard disk, including hard disks arranged in an Redundant Array of Independent Disks (RAID) array, a tape storage drive comprising a magnetic tape data storage device, an optical storage device, Structured Query Language (SQL) servers, cloud technology servers, or the like. In one embodiment, the data storage device 104 may store health related data, such as insurance claims data, consumer data, or the like. The data may be arranged in a database and accessible through SQL queries, or other data base query languages or operations. In some embodiments, data is stored in a secured environment, using one or more security protocols, and retrieved/stored only upon one or more specific user demands.

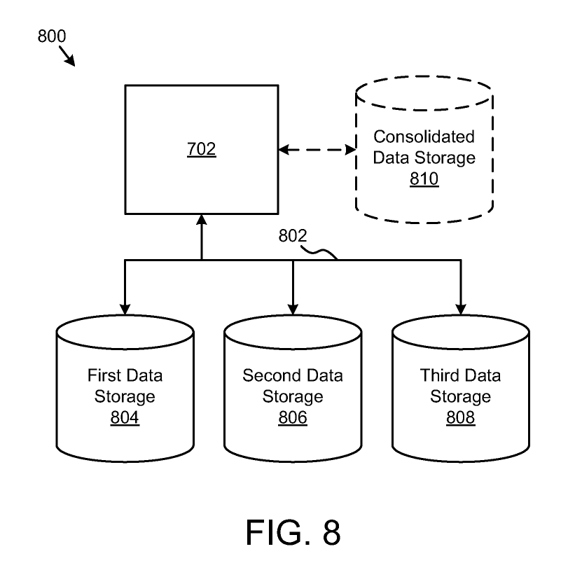

FIG. 8 illustrates one embodiment of a data management system 800 configured to store and manage data for determining a health score. In one embodiment, the system 800 may include a server 802. The server 702 may be coupled to a data-bus 802. In one embodiment, the system 800 may also include a first data storage device 804, a second data storage device 806 and/or a third data storage device 808. In further embodiments, the system 800 may include additional data storage devices (not shown). In such an embodiment, each data storage device 804-808 may host a separate database of individual biometric data, prior health score records and calculations, and/or appeals records. The individual information in each database may be keyed to a common field or identifier, such as an individual's name, social security number, customer number, or the like. Alternatively, the storage devices 804-808 may be arranged in a RAID configuration for storing redundant copies of the database or databases through either synchronous or asynchronous redundancy updates.

In one embodiment, the server 702 may submit a query to selected data storage devices 804-808 to collect a consolidated set of data elements associated with an individual or group of individuals. The server 702 may store the consolidated data set in a consolidated data storage device 810. In such an embodiment, the server 702 may refer back to the consolidated data storage device 810 to obtain a set of data elements associated with a specified individual. Alternatively, the server 702 may query each of the data storage devices 804-808 independently or in a distributed query to obtain the set of data elements associated with a specified individual. In another alternative embodiment, multiple databases may be stored on a single consolidated data storage device 810.

In various embodiments, the server 702 may communicate with the data storage devices 804-810 over the data-bus 802. The data-bus 802 may comprise a SAN, a LAN, or the like. The communication infrastructure may include Ethernet, Fibre-Chanel Arbitrated Loop (FC-AL), Small Computer System Interface (SCSI), and/or other similar data communication schemes associated with data storage and communication. For example, the server 702 may communicate indirectly with the data storage devices 804-810; the server first communicating with a storage server or storage controller 706.

The server 702 may host a software application configured for determining a health score. The software application may further include modules for interfacing with the data storage devices 804-810, interfacing a network 708, interfacing with a user, and the like. In a further embodiment, the server 702 may host an engine, application plug-in, or application programming interface (API). In another embodiment, the server 702 may host a web service or web accessible software application.

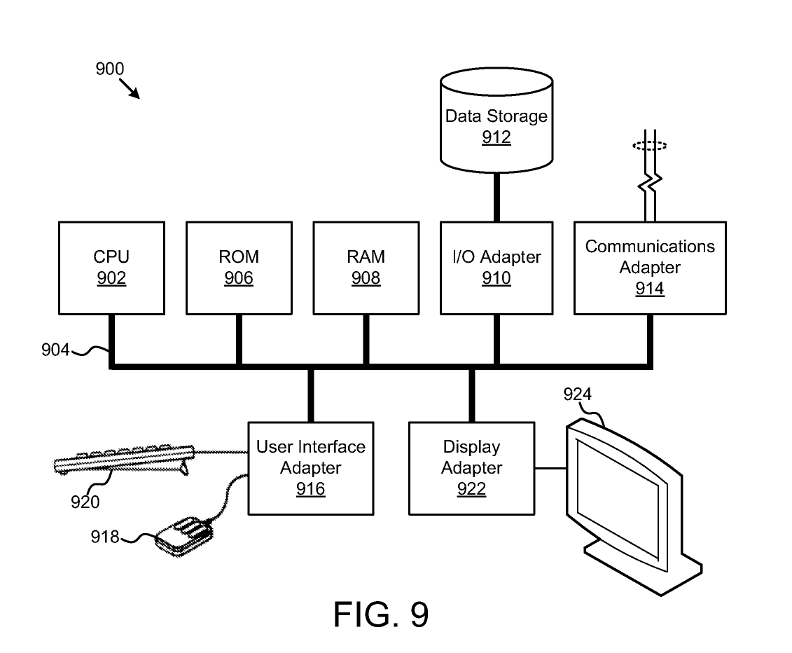

FIG. 9 illustrates a computer system 900 adapted according to certain embodiments of the server 702 and/or the user interface device 710. The central processing unit (CPU) 902 is coupled to the system bus 904. The CPU 902 may be a general purpose CPU or microprocessor. The present embodiments are not restricted by the architecture of the CPU 902, so long as the CPU 902 supports the modules and operations as described herein. The CPU 902 may execute the various logical instructions according to the present embodiments. For example, the CPU 902 may execute machine-level instructions according to the exemplary operations described below with reference to FIGS. 1-6.

The computer system 900 also may include Random Access Memory (RAM) 908, which may be SRAM, DRAM, SDRAM, or the like. The computer system 900 may utilize RAM 908 to store the various data structures used by a software application configured to determine and manage a health score. The computer system 900 may also include Read Only Memory (ROM) 906 which may be PROM, EPROM, EEPROM, optical storage, or the like. The ROM may store configuration information for booting the computer system 900. The RAM 908 and the ROM 906 hold user and system 700 data.

The computer system 900 may also include an input/output (I/O) adapter 910, a communications adapter 914, a user interface adapter 916, and a display adapter 922. The I/O adapter 910 and/or user the interface adapter 916 may, in certain embodiments, enable a user to interact with the computer system 900 in order to input information for choosing where biometric data may be sourced and who may view and/or utilize various health score calculations. In a further embodiment, the display adapter 922 may display a graphical user interface associated with a software or web-based application for determining a health score.

The I/O adapter 910 may connect to one or more storage devices 912, such as one or more of a hard drive, a Compact Disk (CD) drive, a floppy disk drive, a tape drive, to the computer system 900. The communications adapter 314 may be adapted to couple the computer system 900 to the network 706, which may be one or more of a LAN and/or WAN, and/or the Internet. The user interface adapter 916 couples user input devices, such as a keyboard 920 and a pointing device 918, to the computer system 900. The display adapter 922 may be driven by the CPU 902 to control the display on the display device 924.

The present embodiments are not limited to the architecture of system 900. Rather the computer system 900 is provided as an example of one type of computing device that may be adapted to perform the functions of a server 702 and/or the user interface device 710. For example, any suitable processor-based device may be utilized including without limitation, including personal data assistants (PDAs), computer game consoles, and multi-processor servers. Moreover, the present embodiments may be implemented on application specific integrated circuits (ASIC) or very large scale integrated (VLSI) circuits. In fact, persons of ordinary skill in the art may utilize any number of suitable structures capable of executing logical operations according to the described embodiments.

### Managing an Individual and Portable Health Score

FIG. 10 illustrates a block diagram of system 1000 adapted according to certain embodiments of the server 702, data storage 706, and user interface device 710 for managing an individual and portable health score. The embodiments of the systems in FIGS. 8 and 9 as described above with regards to determining a health score may similarly be adapted to manage health score. Generally, system 1000 is configured to selectively receive user healthcare data from one or more different sources, aggregate the received healthcare data, determine a health score, and selectively output the results—the receipt and output of healthcare data controlled by user-inputs.

Healthcare data specific to an individual may be received from a variety of different sources 1002, reflecting various consumption of care choices an individual may have for a given service. For example, healthcare data may be received from one or more health plan sources 1004. Example of health plan sources 1004 include health insurance carriers and health insurance exchanges. Healthcare data may also be received from healthcare providers 1006. In addition to typical healthcare providers 1006 (e.g., doctor's office, clinics, hospitals), healthcare providers 1006 may also include accountable care organizations (ACO) and patient-centered medical homes (PCMH). Healthcare data may also be received from electronic medical records (EMR) vendors 1008 such as for example OptumHealth, Healthvault, WebMD, and the like. Healthcare data may additionally be received from a pharmacy 1010 or a qualified action vendor 1012 (e.g., YMCA or non-smoking clinic). Healthcare data may also be received from government programs 1014 (e.g., Medicare and/or Medicaid). These types of healthcare data sources are provided for example only. One having skill in the art will recognize that these sources may overlap and additional sources for healthcare data may also be relevant.

In some embodiments, the server 702 may programmed to receive healthcare data from one or more healthcare data sources in response to one or more user inputs. A user of system 1000 may have the control to determine which of the healthcare data sources may be used to aggregate that user's healthcare data. In some embodiments, a user may make healthcare data source flow selections—selectively controlling which healthcare data sources (e.g., channels) data may be retrieved from. In some embodiments user control may be received from user interface device by healthcare data source flow module 1016. Healthcare data source flow module may control which healthcare data sources may be used to aggregate a user's healthcare data based on one or more user inputs. As discussed above, in a preferred embodiment, “user control” over the sources used to aggregate a user's health care data does not translate to that a user self-reporting her own health care data. Rather, the received healthcare data may be certified health care data that has been certified by trained and/or certified healthcare providers/sponsors.

In some embodiments, the server 702 may be programmed to aggregate the received healthcare data from the one or more healthcare data sources. Data aggregation 1010 may include compiling the received data into manageable, minable records—for example, a database may be used. In some embodiments, data aggregation 1010 may include removing redundancy within the received user healthcare data. For example, if the user healthcare data is received from multiple sources, there may be duplicate measurements of the same biometrics. Data aggregation may also include resolving anomalies within the received user healthcare data. For example, Individual A may have two different measurements of BMI from two different sources. Resolving the discrepancy may include weighting and/or prioritizing various healthcare data sources higher than others—a BMI measurement made at a doctor's office may be more likely to be accurate than a BMI measurement made at a pharmacy or clinic. In other embodiments, resolving discrepancies may further include weighting/prioritizing more recent measurements higher than older measurements. As discussed earlier with respect to FIG. 6, if any biometric data is incorrect or inaccurate an individual may appeal to correct discrepancies.

In some embodiments, data aggregation 1010 may further include storing various pieces of data—also referred to as records—in data storage 706. In some embodiments, each of the various user inputs and selections may be stored and recorded. Furthermore, in some embodiments, all of the healthcare data from each of the various sources may be stored both in its raw form and also after it was been de-duplicated and resolved. Storing both the raw healthcare data and processed healthcare data may facilitate user understanding of what data affects their health score and where that data comes from. Moreover, storage of this type of data may not only be useful for understanding an individual's health score but also may be utilized in determining whether an appeal should be granted. In some embodiments, each of the appeals and the result of the appeals are further stored. In some embodiments, data may stored with timestamp data and source data. Timestamp data may reflect when data was stored in data storage, and source data may reflect the source of the data: a calculation, a healthcare data source, or the like.

In some embodiments, data aggregation 1010 may further include storing data such as family history information. To the extent that family history information can be shared within the context of applicable laws, an understanding one's family health history could impact the determination of a health score. In some embodiments, for example, the relative weights of one or more health factors may be adjusted. Moreover, those health factors that are more indicative of one's health risks (based on family history information) could improve the predictive value of the score. Thus, an individual with a family history of heart disease may have certain health factors (e.g., BMI, cholesterol, blood pressure) weighted relatively higher than other factors. Additionally, in some embodiments, family history information may be used to help prioritize and/or adjust the amount of points that may be earned back through qualified actions. For example, an individual with a family history of heart disease may get a greater benefit for completing a qualified action related that disease.

In some embodiments of the system 1000, the server 702 may further determine 1020 a health score. Methods for determining a health score are discussed in detail with regards to FIGS. 1-6. As discussed with regards to those methods, in various embodiments of methods for determining a health score, one or more intermediate health scores may be calculated. In some embodiments of system 1000, data aggregation 1010 may further include maintaining a time dependent record of each of the intermediate and final health scores determined. Such a record may enable an individual to view not only how his or her health score changes over time but how the effect of each of the various components that make up the health score changes over time. By storing raw healthcare data, processed healthcare data, health scores, intermediate health scores, health score calculations, appeals and the like individuals can gain full traceability with regards to their health score and healthcare data.

In some embodiments, the server 702 may generate various different outputs 1030. In some embodiments these outputs may include a visualization of an individual's health score. An individual may be able to visualize each of the different steps and health factors used in the calculation of his/her health score. For example, a user-interface may display each of the types of data displayed in Table 5 and Table 6. As such, an individual may be able to analyze the results and learn how best to improve his or her health score.

In some embodiments, individual healthcare data and/or health scores may be output to one or more external sources. These external sources may include health plans, healthcare providers/clinics, and/or vendors. In some embodiments, a health plan (e.g., a health insurance company) may utilize individual health scores and/or the associated healthcare data to provide incentives. For example, if an individual maintains a threshold health score, a health insurance company may provide a health insurance discount or other like bonus (e.g., gift card). Similarly, a health insurance company may additionally provide discounts and/or bonuses for improving a health score or completing a particular qualified action. In some embodiments, healthcare providers and clinics may utilize individual health scores and the associated healthcare data to provide coaching to improve health. For example, a clinic or vendor (e.g., gym) may assist individuals in analyzing their health score and its various components and suggest various behavior modifications to improve their health score (and thereby improve their health). These behavior modifications may include the completion of certified qualified actions, nutrition counseling, and/or other like general health counseling. In some embodiments, health plans and/or providers/clinics may view individual healthcare and/or associated health scores through portals 1050, 1060, 1070.

In some embodiments, the individual users may control who their individual health scores and/or healthcare data may be revealed to. For example, in some embodiments, though an individual's health score may be administered by the health industry, the individual owns his or her own health score. In some embodiments, and an individual may selectively choose to either (1) keep their score their score current—potentially making them eligible for rewards, (2) opt out—keeping their score invisible to health plans, or (3) let their score expire.

As discussed earlier, an individual may control which healthcare data sources are received by system 1000 through healthcare data source flow selections. Similarly, individuals may control which health plans their healthcare data and individual health scores are visible to through one or more health plan data flow selections. Furthermore, individuals may further be able to control which providers and/or clinics have access to their healthcare data and individual health scores through healthcare provider data flow selections using healthcare provider data flow module 1018. Depending on which health plans and providers a particular user is enrolled in a user may be able to determine which health plans and providers can utilize his or her score. In some instances, a user may be enrolled in a particular health plan or utilize a particular provider and choose not to reveal his or her health score to that health plan or that provider.

### Portability of the Health Score

Embodiments of the health score management system presented in FIG. 10 may allow a particular individual's healthcare data and individual health score to be health plan independent. Moreover, the system 1000 may allow connectivity to multiple different health plans and healthcare providers. Specifically, some embodiments, of the methods and systems described are designed around the principle that health should not vary due to plan coverage. With portability, individuals may be able to own and carry their score beyond “temporary” coverage. As such, the individual health score may further be described as portable. The portability of a health score is premised on the principle that regardless of what health plan may be associated with an individual or an employer, the determination of the health score may not change. In some embodiments, the determination of the baseline health score would not change and would be portable between health plans, but specific sponsor programs (e.g., rewards, action points, and the like) may be tied to a given health plan. In some embodiments, sponsor programs (based on qualified actions, condition overrides, and the like) may be standardized across health plans and thus would also be portable.

In some embodiments, an individual can carry their score from one employer to another. For example, in an instance where both health plans have adopted the portable health score scale to manage their wellness programs, individuals would not be hindered from transferring their health score. Thus, if Individual A changes jobs, he can carry his health score from his previous to his new employer. Because health score provides a common currency of measurement, Individual A can become immediately qualified in the new employer's health plan. Thus, the health score may be considered to be independent of a health plan, and rather, the program is there to support and motivate individuals (e.g., through rewards).

Just as an individual can carry one's health score from one employer to another, employers may be able to change health plans and maintain the use of the health scores. Moreover, employers would be able to aggregate and preserve the health score (and the associated data) and carry the health scores to the new health plan. By allowing such an easy transfer, the health score removes the current issues such as health fragmentation that may be caused by health plan sponsor changes and, instead, promotes continuity during such transitions.

Thus, in some embodiments, the system 1000 for managing the individual and portable health score may be configured to transfer calculated health scores between health plans. More specifically, the system may be configured to receive calculated health scores from a health plan 1004 (or other similar source) (e.g., from individuals leaving that health plan) and also may be configured to output calculated health score to a health plan (or other similar source) (e.g., for individuals joining a new plan). Moreover, in addition to the system 1000 may further be configured to transfer the relevant data including any received health care data, processed received health care data, and/or calculations used to determine the one or more health scores.

In some embodiments, the individual will continue to have access to his health score information from the previous three years as stored in the system and also continue to utilize the health score system to monitor his or her health and improve his or her health. Thus regardless of which health plan or healthcare provider are used by an individual, the individual may still be able to monitor and review their health score and have complete traceability with respect to of all historical healthcare data. Furthermore, portability may allow an individual to choose a particular health plan that offers the best programs and rewards for their health life stage or condition and score level.

The value of this portability—of having a the health score act as a common currency of measurement—can help vendors as well. For example, vendors providing various health services (weight loss programs, smoking cessation programs, diabetes maintenance, exercise programs, and the like) can aggregate health scores from a variety of individuals. These vendors can more effectively determine when and how the health of the individuals in their programs improve their health. Moreover, the health score may be used to provide a success measurement of a given vendor program. Through the success measurement (i.e., by measuring the change in health scores of one or more members over time) vendors can themselves optimize their own services to maximize the efficacy of their programs.

In some embodiments, vendors providing health services may isolate a subset of health scores to determine a success measurement for a subset of health scores. For example, if an individual is considering whether to join a particular weight loss program, the weight loss program could demonstrate the value of the weight loss program for individuals with the same sex, similar age, and/or similar health scores. Moreover, a particular sponsor program may even be able to evaluate how various program options were or were not successful for that subset of individuals.

As discussed earlier, the systems and methods for determining and managing a health score may include motivations for an individual to maintain or improve their health score. Such motivation may include incentives such as gift cards or even payroll contribution deductions. Such incentive programs may be configured to be compliant with current healthcare legislation and laws such as the Health Insurance Portability and Accountability Act (HIPAA) and the Patient Protection and Accordable Care Act (PPACA). For example, effective in January 2014, §2705 of the PPACA allows employers to provide financial incentives for participation in wellness programs. Under the recent legislation, employer health plans may be able to provide wellness program participation incentives to plan participants of up to 30 to 50% of the total cost of coverage. In some embodiments, any of the incentives provided to an individual may be provided consistent with these limitations. For example, an individual that improves their health score and participates in various certified qualified actions may receive up to a 30 to 50% payroll contribution deduction.

The methods and systems disclosed here may be configured to support current healthcare legislation in other ways. For example, under §2717 health plans must submit annual reports to the Secretary of Health and Human Services to enable a focus on ways to improve health plans based on case management, disease management, and wellness and health promotion activities. In some embodiments of the disclosed systems and methods, these annual reports may utilize an aggregation of individual health scores to detail the success of various case management, disease management and wellness and health promotion activities used in their health plans. For example, a health plan may be able to demonstrate the efficacy of a particular incentive and wellness program by showing an aggregate increase in individual member health scores.

Likewise, the health score provides a “starter set” of basic measures accepted and used by many organizations known in the art (e.g., HEDIS/NCQA, STARS, USPSTF, and the like). Use of the health score may provide reinforcement to the goals of each those organizations through individual awareness and accountability. Specifically, with adoption of the health score systems and methods disclosed herein, commercial health care payers, providers, ACOs and government programs alike may benefit from better informed and motivated individuals who have the tools they need to take better care of themselves. Additionally, they may benefit from savings associated with administrative efficiencies and more timely deployment of clinical resources, and from increased revenue resulting from improved quality ratings and earlier identification of high risk individuals. Inherently, methods and systems presented in this disclosure may improve administrative efficiencies across the industry while providing a foundation for individual health ownership and a focus on improved quality across all levels of the health care system.

All of the methods disclosed and claimed herein can be made and executed without undue experimentation in light of the present disclosure. While the apparatus and methods of this invention have been described in terms of preferred embodiments, it will be apparent to those of skill in the art that variations may be applied to the methods and in the steps or in the sequence of steps of the method described herein without departing from the concept, spirit and scope of the invention. In addition, modifications may be made to the disclosed apparatus and components may be eliminated or substituted for the components described herein where the same or similar results would be achieved. All such similar substitutes and modifications apparent to those skilled in the art are deemed to be within the spirit, scope, and concept of the invention as defined by the appended claims.

### Data and Analysis of Individual and Portable Health Scores

We evaluated the distribution of portable health scores based on a sample population. The population used for this analysis was a group of over 60,000 individuals working at two national employers who were eligible for incentives for meeting various biometric goals. In some embodiments, the health score is only valid if all loop 1 measures (BMI, smoking status, blood sugar, LDL and systolic blood pressure) were reported. Thus, we excluded individuals from the modeling if any of these measures were not available. Of the roughly 60,000 records, we had full data on 18,047 individuals.

Based on the 18,047 observations for which we had full data, the general characteristics of the population were as follows:
The average age was 44 and 64 percent of the individuals were female
The average BMI was 29
The average fasting blood sugar was 93
The average LDL was 110
The average blood pressure was 120/77 (systolic/diastolic)
All individuals completed a routine physical
80 percent of the females over age 49 had a mammogram
45 percent of the individuals over age 49 had a colorectal screening
72 percent of the females under age 69 had a cervical cancer screening
Approximately 70 percent of the diabetic individuals completed A1C and creatinine tests

A more detailed analysis of the distribution of the 18,047 individuals can be found below in Tables 8 and 9. The embodiment of the health score method used to calculate the health score for this study corresponds closely to the embodiment presented with respect to Tables 3 and 7 described above. In the Tables below, PMPY reflects “per member per year” medical costs. As shown below, through the implementation of the health score, actual medical and health value is being derived. Moreover, as individuals health scores increase, their PMPY medical costs decrease. The adjusted medical costs are adjusted for actuarial factors relative to age, gender and geography. The adjusted medical costs results in a value where the difference is more representative of residual risk of an individual for a given score level, removing the normal effects of aging and the disparity of costs due to geography. The goals of the health score include (1) motivating individuals to improve their own health, and (2) reducing costs. These preliminary studies help demonstrate that both can be achieved.

TABLE 8
Distribution of Individuals (count/% by range)

| Score Range | 250-399 | 400-549 | 550-699 | 700-849 | 850-1,000 |
|---|---|---|---|---|---|
| All | 585/3.2% | 968/5.4% | 2,764/15.3% | 4,685/26.% | 9,045/50.1% |
| With Chronic | 427/14.2% | 426/14.2% | 659/21.9% | 731/24.3% | 767/25.5% |
| Illness Only | | | | | |

TABLE 9
Profile of Individuals by Range

| Score Range | 250-399 | 400-549 | 550-699 | 700-849 | 850-1,000 |
|---|---|---|---|---|---|
| Average age | 52.8 | 49.8 | 47.2 | 43.7 | 41.0 |
| % Age >=50 | 72% | 57% | 44% | 28% | 16% |
| % Female | 78% | 71% | 70% | 64% | 61% |
| % Tobacco | 65% | 60% | 56% | 34% | 0% |
| Use | | | | | |
| Average | 38.6 | 35.6 | 32.3 | 30.2 | 25.2 |
| BMI | | | | | |
| Average | 130 | 109 | 97 | 92 | 88 |
| FBS | | | | | |
| Average | 122 | 121 | 116 | 114 | 104 |
| LDL | | | | | |
| Average | 135 | 131 | 125 | 121 | 117 |
| Systolic BP | | | | | |
| Average | 2.51 | 1.91 | 1.58 | 1.32 | 1.09 |
| Risk | | | | | |
| Score | | | | | |
| PMPY | $6,276 | $4,621 | $3,985 | $3,294 | $2,627 |
| Medical | | | | | |
| Adjusted | $3,484 | $2,756 | $2,585 | $2,398 | $2,132 |
| PMPY | | | | | |
| Medical | | | | | |

What is claimed is:

1. A method for determining a health score comprising:
receiving individual health data;
determining one or more relevant health factors in response to the individual health data;
assigning relative weights to the one or more relevant health factors;
determining, with a processing device, a baseline health score based on the relative weights of the one or more relevant health factors and the individual health data; and
adjusting the baseline health score in response to one or more qualified health actions.

2. The method of claim 1, where the individual health data comprises certified health data.

3. The method of claim 1, where the health score is determined independent of a health plan.

4. The method of claim 1, where the health score in determined independent of a vendor.

5. The method of claim 1, further comprising:
adjusting the baseline health score in response to one or more condition overrides.

6. The method of claim 1, further comprising:
adjusting the baseline health score in response to quality checking.

7. The method of claim 1, where determining one or more relevant health factors comprises determining:
one or more core health factors;
one or more age-gender based factors; and
one or more health condition based factors.

8. The method of claim 1, where determining the baseline health score comprises:
determining a points adjustment for each relevant health factor in response to a result measurement and a target measurement; and
subtracting the points adjustment for each relevant health factor from an initial health score.

9. The method of claim 8, where determining a points adjustment for each relevant health factor comprises:
determining a miss metric by comparing the result measurement to the target measurement;
determining a final difference by comparing the miss metric to a metric cap;
determining a points per increment of the final difference by comparing the relevant health factor's relative weight to the metric cap; and
determining the points adjustment by multiplying the final difference and the points per increment.

10. The method of claim 1, where adjusting the baseline health score in response to one or more certified qualified actions comprises:
determining the applicability of a certified qualified action;
determining the period of applicability of the certified qualified action; and
adjusting the baseline health score during the applicable period of the certified qualified action.

11. The method of claim 5, where adjusting the baseline health score in response to one or more condition overrides comprises:
determining the applicability of a condition override;
determining the period of applicability of the condition override; and
adjusting the incremental impact of one or more relevant health factors during the applicable period in response to the condition override.

12. The method of claim 6, where adjusting the baseline health score in response to quality checking comprises:
receiving an appeal;
determining the applicability of the appeal;
determining the period of applicability of the appeal; and
adjusting the baseline health score during the applicable period of the appeal.

13. A system for determining a health score comprising:
a data storage device configured to store a database comprising one or more records;
a server in data communication with the data storage device suitably programmed to:
receive individual health data;
determine one or more relevant health factors in response to the individual health data;
assign relative weights to the one or more relevant health factors;
determine a baseline health score based on the relative weights of the one or more relevant health factors and the individual health data; and
adjust the baseline health score in response to one or more qualified health actions.

14. The system of claim 13, where the individual health data comprises certified health data.

15. The system of claim 13, where the health score is determined independent of a health plan.

16. The system of claim 13, the server further programmed to:
adjust the baseline health score in response to one or more condition overrides.

17. The system of claim 13, the server further programmed to:
adjust the baseline health score in response to quality checking.

18. The system of claim 13, where determining one or more relevant health factors comprises determining:
one or more core health factors;
one or more age-gender based factors; and
one or more health condition based factors.

19. The system of claim 13, where determining the baseline health score comprises:
determining a points adjustment for each relevant health factor in response to a result measurement and a target measurement; and
subtracting the points adjustment for each relevant health factor from an initial health score.

20. The system of claim 19, where determining a points adjustment for each relevant health factor comprises:
determining a miss metric by comparing the result measurement to the target measurement;
determining a final difference by comparing the miss metric to a metric cap;
determining a points per increment of the final difference by comparing the relevant health factor's relative weight to the metric cap;
determining the points adjustment by multiplying the final difference and the points per increment.

21. The system of claim 16, where adjusting the baseline health score in response to one or more certified qualified actions comprises:
determining the applicability of a certified qualified action;
determining the period of applicability of the certified qualified action; and
adjusting the baseline health score during the applicable period of the certified qualified action.

22. The system of claim 16, where adjusting the baseline health score in response to one or more condition overrides comprises:
determining the applicability of a condition override;
determining the period of applicability of the condition override; and
adjusting the incremental impact of one or more relevant health factors during the applicable period in response to the condition override.

23. The system of claim 17, where adjusting the baseline health score in response to quality checking comprises:
receiving an appeal;
determining the applicability of the appeal;
determining the period of applicability of the appeal; and
adjusting the baseline health score during the applicable period of the appeal.

24. A system for managing one or more health scores comprising:
a data storage device configured to store a database comprising one or more records;
a server in data communication with the data storage device suitably programmed to:
receive one or more user inputs;
receive healthcare data from one or more healthcare data sources in response to the one or more user inputs;
aggregate the received healthcare data, where aggregating comprises:
removing redundancy within the received healthcare data, and
resolving anomalies within the received healthcare data; and
determine a first health score in response to the processed received healthcare data.

25. The system of claim 24, the server further configured to:
determine a plurality of health scores; and
determine a success measurement in response to the plurality of health scores.

26. The system of claim 24, the one or more records comprising:
received healthcare data;
one or more calculated health scores;
one or more calculations used to determine the one or more health scores.

27. The system of claim 26, further configured to:
receive a first set of one or more calculated health scores from a first health plan; and
output a second set of one or more calculated health scores to a second health plan.

28. The system of claim 26, the one or more records comprising:
one or more appeals.

29. The system of claim 28, the one or more records further comprising:
timestamp data describing when the records were stored in the data storage device;
source data describing the source of the records.

30. The system of claim 29, the server further programmed to output one or more records in response to one or more user inputs.

31. The system of claim 24, where receiving one or more user inputs comprises:
receiving one or more healthcare data source flow selections;
receiving one or more healthcare provider data flow selections; and
receiving one more health plan data flow selections.

32. The system of claim 31, the server further configured to control access to one or more records in response to receiving one or more health plan data flow selections.

33. The system of claim 31, the server further configured to output one or more records to a health plan in response to one or more health plan data flow selections.

34. The system of claim 31, where receiving user healthcare data from one or more health data sources comprises receiving user healthcare data across one or more data channels in response to one or more healthcare data source flow selections.

35. A method for determining a health score comprising:
Assigning, with a processing device, relative weights to one or more core health factors, one or more age-gender based factors, and one or more health condition based factors;
determining a first intermediate health score in response to the one or more core factors;
determining a second intermediate health score by adjusting the first intermediate health score in response to the one or more age-gender based factors;
determining a third intermediate health score by adjusting the second intermediate health score in response to the one or more health condition based factors;
determining a fourth intermediate health score by adjusting the third intermediate health score in response to one or more condition overrides;
determining a fifth intermediate health score by adjusting the fourth intermediate health score in response to the one or more qualified health actions; and
determining the health score by adjusting the fifth intermediate health score in response to quality checking.
